SEQUENCE LISTING (96 sequences)

SEQ ID NO: 1 - DNA, 492 bp, Lactococcus lactis
  source 1..492: mol_type = unassigned DNA
ataacatattacttggaagaagaggattttgaaaatcttttttcagaaatgaaacctatagttatgaaattaatgaaacaaattcgcattagaacatggaaaatagaggattatcttcaagaggggatgattattttacatcttctattagaagagcagaacgatggtcaaaagctgcatacaaaatttaaggtaaagtatcatcaaagattaatagatgaattaagacgaagttatgcaaagaaacgaagccatgaccattttataggtttagatgtttatgaatgctcagactggataaattcaggtgatactagtccagataatgaagtggtcttcaatcatttgctggcagaagtatatgaaggtttgagcgcacattatcaagacttactacttcgacaaatgcgaggagaagaactaactcgcatgcaacggtatcgccttcgtgaaaaaataaaggccatcttattttcagaagacgaagagtga

SEQ ID NO: 2 - protein, 163 aa, Lactococcus lactis
MTYYLEEEDFENLFSEMKPIVMKLMKQIRIRTWKIEDYLQEGMIILHLLLEEQNDGQKLHTKFKVKYHQRLIDELRRSYAKKRSHDHFIGLDVYECSDWINSGDTSPDNEVVFNHLLAEVYEGLSAHYQDLLLRQMRGEELTRMQRYRLREKIKAILFSEDEE

SEQ ID NO: 3 - DNA, 492 bp, Lactococcus lactis
  source 1..492: mol_type = unassigned DNA
atgacatattacctggaagaaaatgaattcgaaggtttattttctggaatgaaaccaatcatcagaaaattgatgaaacaaattcgaatcaaagcatgggacatagaggattattatcaagaaggaatgattattttgcatcaccttttagaagaaaatcacccatccactaatatttatacaaagttcaaagtaaaatatcatcaacatttgattgatgaactacgccatagctacgccaaaaaacggcttcatgaccattttgtaggtctggacatttatgaatgttcggactggatagatgcaggaggaagtacccctgaaagcgagcttgtgttcaatcatcttttagcagaagtttatgaaggattgagcgcccactatcaggaattactcgtgcgtcaaatgagaggagaagaactcacgcgaatggaacgctatcggctaagagaaaaaatcaaaaatatactattttctcgagatgatgattaa

SEQ ID NO: 4 - protein, 163 aa, Lactococcus lactis
MTYYLEENEFEGLFSGMKPIIRKLMKQIRIKAWDIEDYYQEGMIILHHLLEENHPSTNIYTKFKVKYHQHLIDELRHSYAKKRLHDHFVGLDIYECSDWIDAGGSTPESELVFNHLLAEVYEGLSAHYQELLVRQMRGEELTRMERYRLREKIKNILFSRDDD

SEQ ID NO: 5 - DNA, 492 bp, Lactococcus lactis
  source 1..492: mol_type = unassigned DNA
atggatgacattcaagaaaaatacggtttagaattcaacgaattattctctgagatgcggccgataatttataaattgatgaagcaattgcacatcaacacatgggattacgatgattacttccaagagggaatgattacactacatgaattgctgcagaaaattacaaatttagatcatgtacatacgaaatttaaagtggcttaccatcagcacttaattgacgaaattcgccatattaaagcacgaaaaagaggttttgatcagctccatccgatcaatgtttatgactgcgcagattggattggctcaaaccttgctacacctgaaagcgagatagttttcaaccatctactagaagaagtttatgataaactttcaacacactataaagaactgttggtaaagcaaatgcatggggaacatcttacgagaatgcagaagtatcgtttaaaggaaaaaattaaagcgattttatttgatgaagactaa

SEQ ID NO: 6 - protein, 163 aa, Lactococcus lactis
MDDIQEKYGLEFNELFSEMRPIIYKLMKQLHINTWDYDDYFQEGMITLHELLQKITNLDHVHTKFKVAYHQHLIDEIRHIKARKRGFDQLHPINVYDCADWIGSNLATPESEIVFNHLLEEVYDKLSTHYKELLVKQMHGEHLTRMQKYRLKEKIKAILFDED

SEQ ID NO: 7 - DNA, 405 bp, Lactococcus raffinolactis
  source 1..405: mol_type = unassigned DNA
atggataaaattgaaaccatacttaaaagtattgaaccgattattatgaactgtcggaaaaaaactaaaattccttcctgggaattagacgactatatgcaggaagggatgattattgctttagagatgtaccatcaactcttattagatccaccagatgatgactttaacttctatgtctatttcaaagtcaggtattcttgtttcttaattgatcactatcgcaaagctatggcagtcaagagaaaattcgaccagcttgactattgtgaactttctgagtctgttaatctttttgatcacaaacaaaatgtgtctgaaaacgtcatgtataacttgttgtgtcaagaaatacacttggttttatccccggaggagctcaagctttttgaggcacttatttga

SEQ ID NO: 8 - protein, 134 aa, Lactococcus raffinolactis
MDKIETILKSIEPIIMNCRKKTKIPSWELDDYMQEGMIIALEMYHQLLLDPPDDDFNFYVYFKVRYSCFLIDHYRKAMAVKRKFDQLDYCELSESVNLFDHKQNVSENVMYNLLCQEIHLVLSPEELKLFEALI

SEQ ID NO: 9 - DNA, 480 bp, Lactococcus plantarum
  source 1..480: mol_type = unassigned DNA
atggatagcatagaaatgatgcttcaaaatattgagccaattattatgaattgtagtaaaacaactaggattccatcttgggagctagatgattacatgcaggaggggatgattattgcactggaaatgtatcaaaatagacataacatcaataacggtaacgcgtttaatttctatgtctattttaaagtcaggtattcctgttacctgatagatagttttagaaaggctaacgcatataaaagaaaatttgatcaaccattatattgtgaaatatctgaagccttcaacctttatgatcaccaccaaaatgttgcagacaatgtctgttatcagctattgcaagttgaaattcttgagatattaacaccagatgaagctgatttatttatgaccttgaaaaatggtgggaaagtagagagaaataaaaagtatagattaaagaaaaaaattattgattatcttaaagacatgttatga

SEQ ID NO: 10 - protein, 159 aa, Lactococcus plantarum
MDSIEMMLQNIEPIIMNCSKTTRIPSWELDDYMQEGMIIALEMYQNRHNINNGNAFNFYVYFKVRYSCYLIDSFRKANAYKRKFDQPLYCEISEAFNLYDHHQNVADNVCYQLLQVEILEILTPDEADLFMTLKNGGKVERNKKYRLKKKIIDYLKDML

SEQ ID NO: 11 - DNA, 480 bp, Lactococcus piscium
  source 1..480: mol_type = unassigned DNA
atggagactttagaagccatgctcaaaaacattgaacctattattatgaattgtcaaaagatggcaaaaataccttcctgggatattgacgattatatgcaggaggggaggatcattgcattagacttgtataatcagctagcagaaagaatggagacggatgaggtgaacttttacgtctacttcaaagtcagatatacctgtttcttgattgatacttaccgtaagacaaatgcctttaaaagaaaatttgaccaaccgatttacttagatgtatccgaagcatttaatctgtatgatcataagcagaatgtcgctgataatgtcatgtatactttattgcatcaggagattctagacatcttaacgcctgtagaaattcaaacgctaaacgcactaaaaaggggagaaaaggtcgaccgcaataaaaaatttaggattaaaaagaagattatcaactatattaatcagattttctag

SEQ ID NO: 12 - protein, 159 aa, Lactococcus piscium
METLEAMLKNIEPIIMNCQKMAKIPSWDIDDYMQEGRIIALDLYNQLAERMETDEVNFYVYFKVRYTCFLIDTYRKTNAFKRKFDQPIYLDVSEAFNLYDHKQNVADNVMYTLLHQEILDILTPVEIQTLNALKRGEKVDRNKKFRIKKKIINYINQIF

SEQ ID NO: 13 - DNA, 486 bp, Lactococcus garvieae
  source 1..486: mol_type = unassigned DNA
atggagcataatttagatatggagcagctggaagaaatttttcattctgtccaacatattgtgtggaagaacagtcgtttgattccgataaatttttggacgtttgatgactatcagcaggaagggcgcttggtattatacgatttgctgggagatggtgtgacgcaaaggaacttattttgccattttaaggtacgctataagcagagacttattgatattaaaagaagggagcgggcttttaaaaggggttttgattgcgggactggcttagatatatacgaatattctgatgctctaaaggggaaagcagccagtccagaacatatcctgatttctggaagtttacttgaagaagtttttgaaaacttaaatttacgctaccgacggctcctcaaaagttacctcgccggcgatgaattgcaccgtatggaaaagtatcgtttgaaggaaaaaataacgaatatattatatgaacagcagtga

SEQ ID NO: 14 - protein, 161 aa, Lactococcus garvieae
MEHNLDMEQLEEIFHSVQHIVWKNSRLIPINFWTFDDYQQEGRLVLYDLLGDGVTQRNLFCHFKVRYKQRLIDIKRRERAFKRGFDCGTGLDIYEYSDALKGKAASPEHILISGSLLEEVFENLNLRYRRLLKSYLAGDELHRMEKYRLKEKITNILYEQQ

SEQ ID NO: 15 - DNA, 489 bp, Lactococcus garvieae
  source 1..489: mol_type = unassigned DNA
atggcagaaaataatttagataaagaacagcttgaagagttattccattcacttcaacatattgtttggaagaacagtcatttaattaaaataaatttttggacaatggatgattatcagcaagaagggcgactggttttataccagttacttgaagatggcgtgacacaggaaaaactattttgccattttaaagtgcgatataagcaacggttgattgatataaaaagacgagaaagagcatttaagcggggttttgattgtggggctggtttagatatatatgagtattctgatgccctgaaaggcaaagctaccagtcctgaatataacttaatttcagttactttacttgaagaggttcatcaaagtttgagtttgagataccgcaatttattggagaatcatctgtcaggagtggagttgcatcgaatggaaaaataccgtttaaaggaaaaaatcaagagaatactctatgaagaagaatga

SEQ ID NO: 16 - protein, 162 aa, Lactococcus garvieae
MAENNLDKEQLEELFHSLQHIVWKNSHLIKINFWTMDDYQQEGRLVLYQLLEDGVTQEKLFCHFKVRYKQRLIDIKRRERAFKRGFDCGAGLDIYEYSDALKGKATSPEYNLISVTLLEEVHQSLSLRYRNLLENHLSGVELHRMEKYRLKEKIKRILYEEE

SEQ ID NO: 17 - DNA, 486 bp, Lactococcus garvieae
  source 1..486: mol_type = unassigned DNA
atggagcataatttagatatggagcagctggaagagatatttcattctgttcaacatattgtatggaagaatagtcgtttgattccgataaatttttggacgatagatgactatcagcaggaagggcgtttggtattatatgatttacttgaggatggtgtgacacaaagaaaacttttttgccattttaaagtacgttataagcagagacttattgatattaaaagaagggagcgggcttttaaaaggggttttgactgtgggactgggctagatatttacgaatattcagatgctttaaaaggaaaagtagccagtccagaacatactctgatttctggcagtttgcttgaagaagttttagaaaacttaaatttacgctaccgtgctcttcttaaaagttaccttgctggtgatgaactgcatcgaatggaaaaacatcgtttgaaagaaaaaataataaaaatattatatgatgaacagtga

SEQ ID NO: 18 - protein, 161 aa, Lactococcus garvieae
MEHNLDMEQLEEIFHSVQHIVWKNSRLIPINFWTIDDYQQEGRLVLYDLLEDGVTQRKLFCHFKVRYKQRLIDIKRRERAFKRGFDCGTGLDIYEYSDALKGKVASPEHTLISGSLLEEVLENLNLRYRALLKSYLAGDELHRMEKHRLKEKIIKILYDEQ

SEQ ID NO: 19 - DNA, 438 bp, Lactococcus fujiensis
  source 1..438: mol_type = unassigned DNA
ttgaaaccgatcgtttcaaaatctatgagaacattaaaaatcaatttttggactacagaggattatcatcaagagggtctaattacattaaatgaaatattaaattcaggatgtaaggagtcacaactatacattcactttaaagtcaaatatcgacaaaagctaatagacgtgattagaaaatcacaggcgcaaaaaagaatctgggataatgcagagagtattgatgtttacgaatctgaaaatcaaattaattccagtaactcaaaccccgaagacataatagtctatgacagtcttgtaaaggaagtaataacaaaattaacaccttcataccggaaactactgaaacgacatctaagaggtgaggatgtgacaaggatggaaaaatacagactgaaggaacgaatcaaacaaattttatttgatggtgattga

SEQ ID NO: 20 - protein, 145 aa, Lactococcus fujiensis
MKPIVSKSMRTLKINFWTTEDYHQEGLITLNEILNSGCKESQLYIHFKVKYRQKLIDVIRKSQAQKRIWDNAESIDVYESENQINSSNSNPEDIIVYDSLVKEVITKLTPSYRKLLKRHLRGEDVTRMEKYRLKERIKQILFDGD

SEQ ID NO: 21 - DNA, 480 bp, Lactococcus chungangensis
  source 1..480: mol_type = unassigned DNA
atggataagattgaaaccatacttaaaaatattgaaccgattatcatgaactgtcgaaaaaaaactaacatcccttcctggcaattagacgactatctccaggaaggcatgattattgctctagagatgtatcatcaacttttattagacccaccagatgatgactttaacttctatgtttatttcaaagtgagatattcttgtttcttgattgatcagtatcggagaaacatggctgtcaaaagaaaattcgaccagattgactattgtgaactatctgaggcgttttatctttttgatcaaaatcaagatgtctctgaaaacgtcatgtataatttgttatgtcaagaaatacacttgcttctatctcctgaagaacgagagctttttgaggcacttaaaaatggacagaagattgaccgtaatcaaaagtttcgtatcaagaagaaaattattgaatatattaagaggttttggtga

SEQ ID NO: 22 - protein, 159 aa, Lactococcus chungangensis
MDKIETILKNIEPIIMNCRKKTNIPSWQLDDYLQEGMIIALEMYHQLLLDPPDDDFNFYVYFKVRYSCFLIDQYRRNMAVKRKFDQIDYCELSEAFYLFDQNQDVSENVMYNLLCQEIHLLLSPEERELFEALKNGQKIDRNQKFRIKKKIIEYIKRFW

SEQ ID NO: 23 - DNA, 414 bp, Lactococcus lactis
  source 1..414: mol_type = unassigned DNA
atgcctacaattaaccaattggtacgcaaacctcgtcgtgctcaagtgactaaatctaaatcaccagcaatgaacgttggctacaacagccgtaaaaaagtacaaactaaacttgcaagcccacaaaaacgtggagtagcaactcgtgttggtacaatgactcctaaaaaacctaactcagcgcttcgtaaattcgcgcgtgtacgtctttcaaaccttatggaagtaacagcgtacatcccaggtatcggacacaacctccaagaacacagtgttgtacttcttcgtggtggacgtgtaaaagaccttccaggggtacgttaccatatcgttcgtggtgcacttgatacagcaggtgtcgctgaccgtaaacaaagccgttctaaatacggtgctaaaaaaccaaaagcttaa

SEQ ID NO: 24 - DNA, 414 bp, Lactococcus lactis
  source 1..414: mol_type = unassigned DNA
atgcctacaattaaccaattggtacgcaaacctcgtcgggctcaagtgactaaatctaaatcaccagcaatgaacgttggctacaacagccgtaaaaaagtacaaactaaacttgcaagcccacaaaaacgtggagtagcaactcgtgttggtacaatgactcctataaaacctaactcagcgcttcgtaaattcgcgcgtgtacgtctttcaaaccttatggaagtaacagcgtacatcccaggtatcggacacaacctccaagaacacagtgttgtacttcttcgtggtggacgtgtaaaagaccttccaggggtacgttaccatatcgttcgtggtgcacttgatacagcaggtgtcgctgaccgtaaacaaagccgttctaaatacggtgctaaaaaaccaaaagcttaa

SEQ ID NO: 25 - DNA, 414 bp, Lactococcus lactis
  source 1..414: mol_type = unassigned DNA
atgcctacaattaaccaattggtacgcaaacctcgtcgtgctcaagtgactaaatctaaatcaccagcaatgaacgttggctacaacagccgtaaaaaagtacaaactaaacttgcaagcccacaaaaacgtggagtagcaactcgtgttggtactatgactcctaaaaaacctaactcagcgcttcgtaaattcgcgcgtgtacgtctttcaaaccttatggaagtaacagcgtacatcccaggtatcggacacaacctccaagaacacagtgttgtacttcttcgtggtggacgtgtaaaagaccttccaggggtacgttaccatatcgttcgtggtgcacttgatacagcaggtgtcgctgaccgtaaacaaagccgttctaaatacggtgctaaaaaaccaaaagcttaa

SEQ ID NO: 26 - DNA, 26 bp, synthetic construct
  misc_feature 1..26: note = Oligonucleotide primer
  source 1..26: mol_type = other DNA
aaaagagctcaattatgaaaaagagg

SEQ ID NO: 27 - DNA, 25 bp, synthetic construct
  misc_feature 1..25: note = Oligonucleotide primer
  source 1..25: mol_type = other DNA
aaaactgcagttaatcatcatctcg

SEQ ID NO: 28 - DNA, 28 bp, synthetic construct
  misc_feature 1..28: note = Oligonucleotide primer
  source 1..28: mol_type = other DNA
aaaagagctcataaaaggagaactttcc

SEQ ID NO: 29 - DNA, 25 bp, synthetic construct
  misc_feature 1..25: note = Oligonucleotide primer
  source 1..25: mol_type = other DNA
aaaactgcagtcactcttcgtcttc

SEQ ID NO: 30 - DNA, 29 bp, synthetic construct
  misc_feature 1..29: note = Oligonucleotide primer
  source 1..29: mol_type = other DNA
atatcaattggtcctcgggatatgataag

SEQ ID NO: 31 - DNA, 19 bp, synthetic construct
  misc_feature 1..19: note = Oligonucleotide primer
  source 1..19: mol_type = other DNA
gactttgaacctcaactcc

SEQ ID NO: 32 - DNA, 32 bp, synthetic construct
  misc_feature 1..32: note = Oligonucleotide primer
  source 1..32: mol_type = other DNA
atagtctcgagtttaagcaattgaatcgctag

SEQ ID NO: 33 - DNA, 42 bp, synthetic construct
  misc_feature 1..42: note = Oligonucleotide primer
  source 1..42: mol_type = other DNA
gcaaaaagtttccaaatttcatactagaatatacgcaatttg

SEQ ID NO: 34 - DNA, 42 bp, synthetic construct
  misc_feature 1..42: note = Oligonucleotide primer
  source 1..42: mol_type = other DNA
caaattgcgtatattctagtatgaaatttggaaactttttgc

SEQ ID NO: 35 - DNA, 34 bp, synthetic construct
  misc_feature 1..34: note = Oligonucleotide primer
  source 1..34: mol_type = other DNA
gcgaaaggatccctattaggtatattccatgtgg

SEQ ID NO: 36 - DNA, 37 bp, synthetic construct
  misc_feature 1..37: note = Oligonucleotide primer
  source 1..37: mol_type = other DNA
gctccctcgagggcggctctgttggattaatatatgg

SEQ ID NO: 37 - DNA, 44 bp, synthetic construct
  misc_feature 1..44: note = Oligonucleotide primer
  source 1..44: mol_type = other DNA
ctttatgtttttggcggatctcatactagaatatacgcaatttg

SEQ ID NO: 38 - DNA, 44 bp, synthetic construct
  misc_feature 1..44: note = Oligonucleotide primer
  source 1..44: mol_type = other DNA
caaattgcgtatattctagtatgagatccgccaaaaacataaag

SEQ ID NO: 39 - DNA, 31 bp, synthetic construct
  misc_feature 1..31: note = Oligonucleotide primer
  source 1..31: mol_type = other DNA
gcgaaaggatccttacaatttgggctttccg

SEQ ID NO: 40 - DNA, 31 bp, synthetic construct
  misc_feature 1..31: note = Oligonucleotide primer
  source 1..31: mol_type = other DNA
aaaacccgggtttaagcaattgaatcgctag

SEQ ID NO: 41 - DNA, 30 bp, synthetic construct
  misc_feature 1..30: note = Oligonucleotide primer
  source 1..30: mol_type = other DNA
aaaacccgggaaataaatggctacaaaatt

SEQ ID NO: 42 - DNA, 29 bp, synthetic construct
  misc_feature 1..29: note = Oligonucleotide primer
  source 1..29: mol_type = other DNA
aaaacggccgttacaatttgggctttccg

SEQ ID NO: 43 - DNA, 31 bp, synthetic construct
  misc_feature 1..31: note = Oligonucleotide primer
  source 1..31: mol_type = other DNA
atagtctcgagaaataaatggctacaaaatt

SEQ ID NO: 44 - DNA, 46 bp, synthetic construct
  misc_feature 1..46: note = Oligonucleotide primer
  source 1..46: mol_type = other DNA
ctttatgtttttggcggatctcatactagactatacgcaaataatc

SEQ ID NO: 45 - DNA, 46 bp, synthetic construct
  misc_feature 1..46: note = Oligonucleotide primer
  source 1..46: mol_type = other DNA
gattatttgcgtatagtctagtatgagatccgccaaaaacataaag

SEQ ID NO: 46 - DNA, 42 bp, synthetic construct
  misc_feature 1..42: note = Oligonucleotide primer
  source 1..42: mol_type = other DNA
agcttcctaatacaacacaattaatattgtgttgtattattg

SEQ ID NO: 47 - DNA, 42 bp, synthetic construct
  misc_feature 1..42: note = Oligonucleotide primer
  source 1..42: mol_type = other DNA
aattcaataatacaacacaatattaattgtgttgtattagga

SEQ ID NO: 48 - DNA, 19 bp, synthetic construct
  misc_feature 1..19: note = Oligonucleotide primer
  source 1..19: mol_type = other DNA
atgcctacaattaaccaat

SEQ ID NO: 49 - DNA, 17 bp, synthetic construct
  misc_feature 1..17: note = Oligonucleotide primer
  source 1..17: mol_type = other DNA
caccgtatttagaacgg

SEQ ID NO: 50 - DNA, 19 bp, synthetic construct
  misc_feature 1..19: note = Oligonucleotide primer
  source 1..19: mol_type = other DNA
atgcctactattaaccaat

SEQ ID NO: 51 - DNA, 17 bp, synthetic construct
  misc_feature 1..17: note = Oligonucleotide primer
  source 1..17: mol_type = other DNA
taccgtatttagaacgg

SEQ ID NO: 52 - DNA, 21 bp, synthetic construct
  misc_feature 1..21: note = Oligonucleotide primer
  source 1..21: mol_type = other DNA
agtagtatcagcactgacagc

SEQ ID NO: 53 - DNA, 19 bp, synthetic construct
  misc_feature 1..19: note = Oligonucleotide primer
  source 1..19: mol_type = other DNA
acacctttgttcttgaagg

SEQ ID NO: 54 - DNA, 30 bp, synthetic construct
  misc_feature 1..30: note = Oligonucleotide primer
  source 1..30: mol_type = other DNA
aaagagctcaaaataaaaatgaaattatgg

SEQ ID NO: 55 - DNA, 29 bp, synthetic construct
  misc_feature 1..29: note = Oligonucleotide primer
  source 1..29: mol_type = other DNA
aaagctagcgggaaaaaattgtgaattac

SEQ ID NO: 56 - DNA, 32 bp, synthetic construct
  misc_feature 1..32: note = Oligonucleotide primer
  source 1..32: mol_type = other DNA
aaaaactagtgcagtttaaattcggtcctcgg

SEQ ID NO: 57 - DNA, 31 bp, synthetic construct
  misc_feature 1..31: note = Oligonucleotide primer
  source 1..31: mol_type = other DNA
aaaaactagtgtacagtcggcattatctcat

SEQ ID NO: 58 - DNA, 20 bp, synthetic construct
  misc_feature 1..20: note = Oligonucleotide primer
  source 1..20: mol_type = other DNA
ctttaatgatggaatgattg

SEQ ID NO: 59 - DNA, 47 bp, synthetic construct
  misc_feature 1..47: note = Oligonucleotide primer
  source 1..47: mol_type = other DNA
ctattaatcttatcatatcccgaggatccatataactatatgaaacc

SEQ ID NO: 60 - DNA, 26 bp, synthetic construct
  misc_feature 1..26: note = Oligonucleotide primer
  source 1..26: mol_type = other DNA
tcctcgggatatgataagattaatag

SEQ ID NO: 61 - DNA, 27 bp, synthetic construct
  misc_feature 1..27: note = Oligonucleotide primer
  source 1..27: mol_type = other DNA
tctcatattataaaagccagtcattag

SEQ ID NO: 62 - DNA, 51 bp, synthetic construct
  misc_feature 1..51: note = Oligonucleotide primer
  source 1..51: mol_type = other DNA
ctaatgactggcttttataatatgagacttagaaaaatctaaatatggttg

SEQ ID NO: 63 - DNA, 24 bp, synthetic construct
  misc_feature 1..24: note = Oligonucleotide primer
  source 1..24: mol_type = other DNA
gaagatttttaatttcaagtgtag

SEQ ID NO: 64 - DNA, 20 bp, synthetic construct
  misc_feature 1..20: note = Oligonucleotide primer
  source 1..20: mol_type = other DNA
tcagtaccgaaaaacgaatg

SEQ ID NO: 65 - DNA, 19 bp, synthetic construct
  misc_feature 1..19: note = Oligonucleotide primer
  source 1..19: mol_type = other DNA
atttaccagttccgttagg

SEQ ID NO: 66 - DNA, 20 bp, synthetic construct
  misc_feature 1..20: note = Oligonucleotide primer
  source 1..20: mol_type = other DNA
taacaatgatacagaagatg

SEQ ID NO: 67 - DNA, 46 bp, synthetic construct
  misc_feature 1..46: note = Oligonucleotide primer
  source 1..46: mol_type = other DNA
ctattaatcttatcatatcccgaggatatttttgtcttgtactagg

SEQ ID NO: 68 - DNA, 47 bp, synthetic construct
  misc_feature 1..47: note = Oligonucleotide primer
  source 1..47: mol_type = other DNA
ctaatgactggcttttataatatgagagagagaaaaaaattactgac

SEQ ID NO: 69 - DNA, 20 bp, synthetic construct
  misc_feature 1..20: note = Oligonucleotide primer
  source 1..20: mol_type = other DNA
aaaatctgttagaactgttg

SEQ ID NO: 70 - DNA, 20 bp, synthetic construct
  misc_feature 1..20: note = Oligonucleotide primer
  source 1..20: mol_type = other DNA
aagataaggcagttgaaatg

SEQ ID NO: 71 - DNA, 20 bp, synthetic construct
  misc_feature 1..20: note = Oligonucleotide primer
  source 1..20: mol_type = other DNA
tcaccatgtgaataaagtcc

SEQ ID NO: 72 - DNA, 19 bp, synthetic construct
  misc_feature 1..19: note = Oligonucleotide primer
  source 1..19: mol_type = other DNA
caaaaatgtgaagcttatc

SEQ ID NO: 73 - DNA, 42 bp, synthetic construct
  misc_feature 1..42: note = Oligonucleotide primer
  source 1..42: mol_type = other DNA
ctattaatcttatcatatcccgaggatgcataattcgatttc

SEQ ID NO: 74 - DNA, 45 bp, synthetic construct
  misc_feature 1..45: note = Oligonucleotide primer
  source 1..45: mol_type = other DNA
taatgactggcttttataatatgagactatttatctgctcatttc

SEQ ID NO: 75 - DNA, 19 bp, synthetic construct
  misc_feature 1..19: note = Oligonucleotide primer
  source 1..19: mol_type = other DNA
gagcttttttcaaatcttc

SEQ ID NO: 76 - DNA, 18 bp, synthetic construct
  misc_feature 1..18: note = Oligonucleotide primer
  source 1..18: mol_type = other DNA
gaagtgatgaatgagatg

SEQ ID NO: 77 - DNA, 19 bp, synthetic construct
  misc_feature 1..19: note = Oligonucleotide primer
  source 1..19: mol_type = other DNA
ctttctcatcaattgagac

SEQ ID NO: 78 - DNA, 19 bp, synthetic construct
  misc_feature 1..19: note = Oligonucleotide primer
  source 1..19: mol_type = other DNA
ctttgggttctaatttatc

SEQ ID NO: 79 - DNA, 44 bp, synthetic construct
  misc_feature 1..44: note = Oligonucleotide primer
  source 1..44: mol_type = other DNA
ctattaatcttatcatatcccgaggacgttggtgtatattttac

SEQ ID NO: 80 - DNA, 46 bp, synthetic construct
  misc_feature 1..46: note = Oligonucleotide primer
  source 1..46: mol_type = other DNA
ctaatgactggcttttataatatgagatagaaataaaggaaaggac

SEQ ID NO: 81 - DNA, 19 bp, synthetic construct
  misc_feature 1..19: note = Oligonucleotide primer
  source 1..19: mol_type = other DNA
ttgctttaaggatagtttc

SEQ ID NO: 82 - DNA, 20 bp, synthetic construct
  misc_feature 1..20: note = Oligonucleotide primer
  source 1..20: mol_type = other DNA
agaagccaataatgacgatg

SEQ ID NO: 83 - DNA, 22 bp, synthetic construct
  misc_feature 1..22: note = Oligonucleotide primer
  source 1..22: mol_type = other DNA
agaattctgatgatgcacagtc

SEQ ID NO: 84 - DNA, 27 bp, synthetic construct
  misc_feature 1..27: note = Oligonucleotide primer
  source 1..27: mol_type = other DNA
agcgccgcggtgggatcctctagagtc

SEQ ID NO: 85 - DNA, 31 bp, synthetic construct
  misc_feature 1..31: note = Oligonucleotide primer
  source 1..31: mol_type = other DNA
ctgcaggcatgcacatcatcaacttgaaggg

SEQ ID NO: 86 - DNA, 30 bp, synthetic construct
  misc_feature 1..30: note = Oligonucleotide primer
  source 1..30: mol_type = other DNA
cccaccgcggtggagatacgaacaaattag

SEQ ID NO: 87 - DNA, 27 bp, synthetic construct
  misc_feature 1..27: note = Oligonucleotide primer
  source 1..27: mol_type = other DNA
gatagtaactccttaatttttatttgc

SEQ ID NO: 88 - DNA, 58 bp, synthetic construct
  misc_feature 1..58: note = Oligonucleotide primer
  source 1..58: mol_type = other DNA
gcaaataaaaattaaggagttactatcatgacatattacttggaagaagaggattttg

SEQ ID NO: 89 - DNA, 45 bp, synthetic construct
  misc_feature 1..45: note = Oligonucleotide primer
  source 1..45: mol_type = other DNA
ccttcaagttgatgatgtgcatgcctgcagtcactcttcgtcttc

SEQ ID NO: 90 - DNA, 27 bp, synthetic construct
  misc_feature 1..27: note = Oligonucleotide primer
  source 1..27: mol_type = other DNA
tgaccatgttacacaagcctatatcct

SEQ ID NO: 91 - DNA, 56 bp, synthetic construct
  misc_feature 1..56: note = Oligonucleotide primer
  source 1..56: mol_type = other DNA
cgcccttatgggatttatcttccttacttcgtttctttgcataacttcgtcttaat

SEQ ID NO: 92 - DNA, 23 bp, synthetic construct
  misc_feature 1..23: note = Oligonucleotide primer
  source 1..23: mol_type = other DNA
taaggaagataaatcccataagg

SEQ ID NO: 93 - DNA, 23 bp, synthetic construct
  misc_feature 1..23: note = Oligonucleotide primer
  source 1..23: mol_type = other DNA
ttcacgttactaaagggaatgta

SEQ ID NO: 94 - DNA, 57 bp, synthetic construct
  misc_feature 1..57: note = Oligonucleotide primer
  source 1..57: mol_type = other DNA
tctacattccctttagtaacgtgaaccatgaccattttataggtttagatgtttatg

SEQ ID NO: 95 - DNA, 24 bp, synthetic construct
  misc_feature 1..24: note = Oligonucleotide primer
  source 1..24: mol_type = other DNA
cggtgttcctccatatatctacgc

SEQ ID NO: 96 - DNA, 27 bp, synthetic construct
  misc_feature 1..27: note = Oligonucleotide primer
  source 1..27: mol_type = other DNA
cgctaaactcaacaggtgatccgattg

(19) United States
(12) Patent Application Publication
(10) Pub. No.: US 2025/0388933 A1
(43) Pub. Date: Dec. 25, 2025

(54) METHODS AND STRAIN

(71) Applicant: INTERNATIONAL N&H DENMARK APS, KONGENS LYNGBY (DK)

(72) Inventors: Christophe Fremaux, Poitiers (FR); Philippe Horvath, Chatellerault (FR); Patrick Boyaval, La Meziere (FR); Pascal Hols, Vedrin (BE); David Blandine, Floreffe (BE); Amandine Radziejwoski, Sterrebeek (BE); Laetitia Fontaine, Louvain-la-Neuve (BE); Frederic Toussaint, Rixensart (BE)

(21) Appl. No.: 19/176,263

(22) Filed: Apr. 11, 2025

Related U.S. Application Data
(62) Division of application No. 18/112,968, filed on Feb. 22, 2023, now Pat. No. 12,297,448.
Application No. 18/112,968 is a continuation of application No. 16/469,784, filed on Jun. 14, 2019, now abandoned.

(30) Foreign Application Priority Data
Dec. 19, 2016 (EP) 16205055.3

(51) Int. Cl. C12N 15/90; C07K 7/00; C12Q 1/02

(52) U.S. Cl. CPC C12N 15/902; C07K 7/00; C12Q 1/02

(57) ABSTRACT

The present invention relates to a method for transforming a strain of the Lactococcus genus through natural competence. The present invention further relates to strains obtained or obtainable by said method. The present invention also relates to a method for identifying a strain of the Lactococcus genus which is transformable through natural competence.

### CROSS-REFERENCE TO RELATED APPLICATIONS

This application is a continuation of U.S. patent application Ser. No. 16/469,784, filed Jun. 14, 2019, which is a § 371 U.S. national stage patent application of PCT Patent Application No. PCT/EP2017/083601, filed Dec. 19, 2017, which claims priority to European Patent Application No. 16205055.3, filed Dec. 19, 2016, the contents of each which are hereby incorporated by reference in their entireties.

### REFERENCE TO A SEQUENCE LISTING

The contents of the electronic submission of the text file Sequence Listing, named “NB41019USCNT_SequenceListing.xml” was created on Jun. 21, 2023, and is 119 KB in size, which is hereby incorporated by reference in its entirety.

### FIELD OF THE INVENTION

The present invention relates to a method for transforming a strain of the Lactococcus genus through natural competence. The present invention further relates to strains obtained or obtainable by said method. The present invention also relates to a method for identifying a strain of the Lactococcus genus which is transformable through natural competence.

### BACKGROUND TO THE INVENTION

Lactococcus lactis is one of the most important lactic acid bacteria used in the dairy industry, in particular as a main dairy starter species in various cheese preparations (e.g. gouda, cheddar, brie, parmesan, roquefort) and fermented milk products (e.g. buttermilk, sour cream). Other applications of L. lactis bacteria include as a host for heterologous protein production or as a delivery platform for therapeutic molecules. While the growth and fermentation properties of L. lactis have been gradually improved by selection and classical methods, there is great potential for further improvement through natural processes or by genetic engineering. Of particular interest are methods to naturally transform L. lactis without the use of genetic engineering, thereby generating new non-GMO strains with useful industrial properties.

Lactococcus raffinolactis is present in a wide range of environments, such as foods (meat, fish, milk, vegetable), animals, and plant materials. In the dairy environment, this species has been found in raw milks (cow, ewe, goat, and camel), natural dairy starter cultures, and a great variety of cheeses. The prevalence of this bacterium in foods even if with a “nondominant” status compared to other lactococci could make it a candidate for future development of starter cultures.

DNA acquisition by natural transformation is widespread among prokaryotes and has been identified in over 80 species. Various functions are attributed to competence for natural transformation: genome plasticity, DNA repair, and/or nutrition. In Gram-positive bacteria, competence for natural transformation has been well-characterized in Bacillus subtilis and in various species of the genus Streptococcus (e.g. S. pneumoniae, S. mutans, and S. thermophilus).

In streptococci, competence for DNA transformation is induced in response to secreted signalling peptides referred to as competence pheromones/alarmones. The production of this class of cell-to-cell communication molecules is initiated in response to specific environmental stresses or conditions and allows the coordination of physiological functions (e.g. competence, predation, biofilm formation). Above a threshold concentration, competence pheromones activate the master regulator ComX (alternative sigma factor x), which ultimately leads to a transcriptional reprogramming of cells (globally known as late competence phase) including the induction of genes strictly required for DNA transformation. ComX binds to a specific DNA sequence named Com-box or Cin-box, which is located at least in the vicinity of promoters of late competence (com) genes/operons responsible for DNA uptake (e.g.; comG, comF and comE operons), DNA protection (e.g. ssb) and DNA recombination (e.g. recA, dprA, coiA), and positively controls their expression.

The early steps leading to competence activation (early competence phase) differs among bacteria. In streptococci, two major peptide-based signaling pathways—i.e. ComCDE and ComRS—have been identified so far. In mitis and anginosus groups of streptococci (S. pneumoniae as paradigm), the competence signaling peptide (CSP, or mature ComC) triggers a phosphorylation cascade mediated by the two-component system ComD-ComE, leading to the transcriptional activation of comX. In salivarius, mutans, pyogenes, bovis and suis groups of streptococci, another regulation mechanism is operational (S. thermophilus as paradigm). This system involves the ComX-induction peptide (XIP, or mature ComS) which is internalized by the oligopeptide transporter Opp, binds to and activates the regulator ComR, and in turn induces comX transcription.

Orthologues of comX and of all late com genes essential for natural transformation have been identified in the genome of L. lactis, although some are present as putative pseudogenes in different strains (Wydau et al., 2006).

Specific growth conditions have been reported to activate com genes in Lactococcus lactis. For example, the promoter of comX was shown to be induced during cheese-making conditions in strain MG5267 (an MG1363 derivative) which belongs to the subspecies cremoris (Bachmann et al. 2010).

In the L. lactis subspecies (subsp.) lactis, carbon starvation was shown to activate six late com genes in strain IL1403 of dairy origin (i.e. comX, comEA, comGA, comGB, radA, and nucA) and most of the late essential com genes in strain KF147 of plant origin (i.e. comX, comC, coiA, and operons comG, comE, comF) (Ercan et al., 2015). However, when the authors attempted to validate functional natural transformation in KF147, they were unsuccessful.

Wydau et al. reported that all the well-established late genes/operons display an upstream and conserved Com-box, suggesting that they are similarly controlled by ComX as reported in streptococci. However, the authors did not comment on whether comX over-expression in IL1403 induced natural competence. Indeed, the authors neither report any experiment evaluating natural competence in this strain nor suggest any experimental conditions appropriate for inducing natural competence. Thus, as noted in the recent literature (see Ercan et al., 2015) [i.e., 9 years after Wydau et al.], there is no experimental evidence for successful transformation of any species of the genus Lactococcus by natural competence, and even less of IL1403.

Accordingly, there remains a need for a method for naturally transforming Lactococcus strains using natural competence. In addition, since some strains of the Lactococcus genus may not encode a full set of functional late com genes, there is a need for a method for identifying Lactococcus strains which can be transformed by natural competence.

### SUMMARY OF THE INVENTION

In a first aspect, the present invention provides a method for transforming a strain of the Lactococcus genus with an exogenous DNA polynucleotide comprising the steps of:
(a) providing a strain of the Lactococcus genus, wherein said strain is transformable through natural competence;
(b) modulating the production of a ComX protein in said strain;
(c) contacting said strain of step (b) with an exogenous DNA polynucleotide in a medium and incubating the resulting mixture for integration of the exogenous DNA polynucleotide into the genome of said strain; and
(d) selecting a strain which has integrated the exogenous DNA polynucleotide into its genome.

In one embodiment, the step of modulating the production of a ComX protein is performed by expressing a comX gene in said strain or increasing the expression of a comX gene in said strain.

In a further embodiment, the comX gene is an exogenous comX gene. Said exogenous comX gene may be transferred into said strain by conjugation, transduction, or transformation. Said exogenous comX gene may be operably linked to transcription regulator(s).

In an alternative embodiment, said comX gene is the endogenous comX gene of said strain.

In one embodiment, when said comX gene is the endogenous comX gene of said strain, the method comprises:
(a) providing a strain of the Lactococcus genus, wherein said strain is transformable through natural competence;
(b) modulating the production of a ComX protein, by expressing the endogenous comX gene or increasing the expression of the endegenous comX of said strain;
(c) contacting said strain of step (b) with an exogenous DNA polynucleotide in a medium and incubating the resulting mixture for integration of the exogenous DNA polynucleotide into the genome of said strain; and
(d) selecting a strain which has integrated the exogenous DNA polynucleotide into its genome,
wherein step (c) is carried out after step (b) or wherein step (b) and step (c) are carried out simultaneously.

In some embodiments, said ComX protein has the amino acid sequence of SEQ ID NO:2, SEQ ID NO: 4, SEQ ID NO:6, SEQ ID NO:8, SEQ ID NO:10, SEQ ID NO:12, SEQ ID NO:14, SEQ ID NO: 16, SEQ ID NO:18, SEQ ID NO:20, SEQ ID NO:22, or has at least 90% identity or at least 90% similarity to the amino acid sequence of SEQ ID NO:2, SEQ ID NO:4, SEQ ID NO:6, SEQ ID NO:8, SEQ ID NO:10, SEQ ID NO:12, SEQ ID NO:14, SEQ ID NO:16, SEQ ID NO:18, SEQ ID NO:20 or SEQ ID NO:22. In some embodiments, said ComX protein has the amino acid sequence of SEQ ID NO:2, SEQ ID NO:4, SEQ ID NO:6, or has at least 90% identity or at least 90% similarity to the amino acid sequence of SEQ ID NO:2, SEQ ID NO:4 or SEQ ID NO:6.

In some embodiments, said comX gene has the nucleotide sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO: 17, SEQ ID NO: 19, SEQ ID NO:21, or has at least 90% identity to the nucleotide sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO:19 or SEQ ID NO:21.

In some embodiments, said comX gene has the nucleotide sequence of SEQ ID NO:1, SEQ ID NO: 3, SEQ ID NO:5, or has at least 90% identity to the nucleotide sequence of SEQ ID NO: 1, SEQ ID NO:3 or SEQ ID NO:5.

In some embodiments, the medium of step (c) is a chemically defined medium. In a preferred embodiment the chemically defined medium (CDM) comprises 0.5 g/L NH₄Cl, 9.0 g/L KH₂PO₄, 7.5 g/L K₂HPO₄, 0.2 g/L MgCl₂, 5 mg/L FeCl₂, 50 mg/L CaCl₂), 5 mg/L ZnSO₄, 2.5 mg/L CoCl₂, 0.05 g/L tyrosine, 0.1 g/L asparagine, 0.1 g/L cysteine, 0.1 g/L glutamine, 0.1 g/L isoleucine, 0.1 g/L leucine, 0.1 g/L methionine, 0.1 g/L tryptophan, 0.1 g/L valine, 0.1 g/L histidine, 0.2 g/L arginine, 0.2 g/L glycine, 0.2 g/L lysine, 0.2 g/L phenylalanine, 0.2 g/L threonine, 0.3 g/L alanine, 0.3 g/L proline, 0.3 g/L serine, 10 mg/L paraaminobenzoic acid, 10 mg/L biotin, 1 mg/L folic acid, 1 mg/L nicotinic acid, 1 mg/L panthotenic acid, 1 mg/L riboflavin, 1 mg/L thiamine, 2 mg/L pyridoxine, 1 mg/L cyanocobalamin, 5 mg/L orotic acid, 5 mg/L 2-deoxythymidine, 5 mg/L inosine, 2.5 mg/L dl-6,8-thioctic acid, 5 mg/L pyridoxamine, 10 mg/L adenine, 10 mg/L guanine, 10 mg/L uracil, 10 mg/L xanthine, and 5 g/L glucose.

In some embodiments, prior to step (c) said strain is incubated in a pre-culture medium, preferably wherein the pre-culture medium is a complex medium, more preferably wherein the pre-culture medium is M17G or THBG.

In some embodiments of the present invention, said strain is incubated with the exogenous DNA polynucleotide for around 4 to 8 hours at around 30° C. and said medium of step (c) is supplemented with an osmo-stablizer, preferably wherein the osmo-stablizer is glycerol or mannitol, more preferably wherein the osmo-stabilizer is 5% [v/v] glycerol or 5% [w/v] mannitol.

In some embodiments, said exogenous DNA polynucleotide is from a strain of the Lactococcus lactis species.

In some embodiments, said exogenous DNA polynucleotide is from a strain of the Lactococcus raffinolactis species.

In some embodiments, said strain of step (a) is a Lactoccocus lactis subsp. cremoris strain.

In another aspect, the present invention provides a strain of the Lactococcus genus obtained or obtainable by the method of the first aspect of the present invention.

In one embodiment, said strain of the Lactococcus genus is a strain of the Lactococcus lactis or Lactococcus raffinolactis species.

In a further aspect, the present invention provides a method for identifying a strain of the Lactococcus genus which is transformable through natural competence comprising the steps of:
(a) providing a strain of the Lactococcus genus species;
(b) transforming said strain with a plasmid expressing a comX gene having at least 90% identity, preferably having 100% identity, to the endogenous comX gene of said strain;
(c) contacting said strain obtained in step (b) with an exogenous DNA polynucleotide encoding a marker gene in a medium and incubating the resulting mixture for integration of the exogenous DNA polynucleotide into the genome of said strain; and
(d) determining the rate of recombination events;
   wherein a rate of at least 1×10⁻⁶ transformants per μg of DNA is indicative of a strain which is transformable through natural competence.

In a particular embodiment of method for transforming a strain of the Lactococcus genus of the present invention, said strain of step (a) is identified using the method for identifying a strain of the Lactococcus genus which is transformable through natural competence according to the present invention. In some embodiments of the present invention, said strain of step (a) is identified using Assay A.

### DESCRIPTION OF THE DRAWINGS

FIG. 1: Table showing the status of genes involved in natural competence for L. lactis strains MG1363, SK11, KW2, IL1403, SL12651 and SL12653.

Late com genes in the complete genomes of strains MG1363, SK11, and KW2 of Lactococcus lactis subsp. cremoris and of strain IL1403, SL12651 and SL12653 of Lactococcus lactis subsp. lactis. Origin is indicated above strain names. Gene-associated function in DNA transformation is indicated on the left. Reg. denotes regulation. The complete and incomplete status of late genes i is based on blastp and tblastn homology searches (https://blast.ncbi.nlm.nih.gov/Blast.cgi) using orthologues of S. pneumoniae TIGR4 and S. thermophilus LMD-9 and default parameters. +denotes the presence of a complete gene; * denotes the presence of an incomplete gene due to nucleotide(s) exchange, insertion or deletion resulting in a premature stop codon; and Tn denotes a disrupted gene by the insertion of at least one transposon.

FIG. 2: Graphs displaying the results of luciferase assays which demonstrate the activation of a reporter construct comprising the late promoter P_{comGA} driven by constitutive comX overexpression

(FIG. 2A) Maximum specific luciferase (Lux) activity (RLU OD₆₀₀⁻¹) emitted by eight independent clones (cl01 to cl08) of the KW2-derived reporter strain (BLD101, P_{comGA[MG]-lux}AB) carrying plasmid pGhP32comX_{MG} compared to the control strain (Ctl) carrying the empty vector pG⁺host9. (FIG. 2B) Kinetics of specific Lux activity (solid line) during growth (RLU/OD600; dotted line) for the control strain (Ctl; black lines) and three selected clones (BLD101 [pGhP32comX_{MG}], cl02, cl04 and cl05; gray lines). (FIG. 2C) Kinetics of specific luciferase activity (closed symbols) during growth (RLU/OD600; open symbols) of the MG1363+pGhP32comX_{MG}-P_{comGA[MG]}-luc, grown in M17G at 30° C. (FIG. 2D) Kinetics of specific luciferase activity (closed symbols) during growth (RLU/OD600; open symbols) of IL1403+pGhP32comX_{IO}-P_{comGA[IO]}-luc strains, grown in M17G at 30° C.

FIG. 3: Graphs Displaying the Results of Luciferase Assays which Demonstrate the Impact of Growth Medium on P_{comGA} Activation

Maximum specific Lux activity of BLD101 [pGhP32comX_{MG}] cl02 grown in different final culture media (CDM, THBG, and M17G) according to preculture conditions (CDM, THBG, and M17G). Overnight precultures were 10-fold diluted in the pre-culture medium and grown for 2 hours. Then, cells were washed twice in distilled water and the OD600 was adjusted to 0.05 in the final growth medium before measuring growth and luciferase activity. One representative experiment of two independent replicates.

FIG. 4: Results of a transformation assay implemented on a L. lactis subsp. cremoris KW2 constitutively expressing comX contacted with a DNA consisting of a mutated allele of the rpsL gene as exogenous DNA polynucleotide (FIG. 4A) Alignment of the rpsL gene sequences of strain MG1363, a spontaneous streptomycin-resistant clone of strain MG1363, strain KW2 and a KW2-derived transformant obtained using the method of the invention (partial sequence). The arobase, pound and dollar signs below the alignment indicate the positions of nucleotide differences existing between the rpsL sequences. The dollar sign at position 167 indicates the point mutation (A→T; strA1 allele) responsible for the streptomycin-resistance phenotype; the pound sign at position 156 highlights a nucleotide that is naturally different between MG1363 and KW2 (T in KW2, A in MG1363); the arobase sign at position 39 indicates a silent nucleotide substitution (T→G) which is found in the streptomycin-resistant clone derived from MG1363. (FIG. 4B) DNA transformation with the strA1 allele was assessed for L. lactis strains constitutively expressing ComX. Transformation rate (white bars) and maximum specific luciferase (Lux) activity (black diamonds, RLU OD₆₀₀⁻¹, as reported in FIG. 2) of eight clones (cl01 to cl08) of the reporter strain (BLD101, P_{comGA[MG]}-luxAB) carrying plasmid pGhP32comX_{MG} compared to the negative control strain (Ctl−) carrying the empty vector (BLD101 [pG⁺host9]).

FIG. 5: Graphs displaying the results of transformation rate of the KW2 derivative BLD101 [pGhP32comX_{MG}] obtained with overlap PCR products (comEC, mecA, ciaRH, covRS and clpC) and strA1 (rpsL*)-donor DNA.

The threshold represents the theoretical transformation rate to obtain only one transformant.

FIG. 6: Graphs depicting the results of transformation assays for a L. lactis subsp. cremoris deleted in its comEC gene and constitutively expressing comX.

DNA transformation with the strA1 allele was assessed for L. lactis strains constitutively expressing comX. Transformation rate (white bars) and maximum specific luciferase (Lux) activity (RLU OD₆₀₀⁻¹) of four clones (cl01 to cl04) of the ComEC-deficient reporter strain (BLD102, P_{comGA[MG]-lux}AB) carrying plasmid pGhP32comX_{MG} compared to the positive (Ctl⁺, BLD101 [pGhP32comX_{MG}] cl02) and negative (Ctl, BLD101 [pG⁺host9]) control strains. Transformability was assessed according to the standard protocol described in Materials and Methods using strA1-carrying PCR products as donor DNA. ND denotes a transformation rate below the detection level of spontaneous Str′ mutants (<10⁻⁷). One representative experiment of two independent replicates.

FIG. 7: Graphs displaying natural competence in Lactococcus lactis subsp. lactis SL12651 and 12653 strains.

(FIG. 7A) Transformation rate of L. lactis subsp. lactis SL12651 and 12653 strains in M17G medium, with rpsL* donor DNA (+DNA) or without donor DNA (−DNA); (FIG. 7B) DNA transformation with increasing initial concentration of donor DNA assessed in SL12653 strain; (FIG. 7C) Comparison of transformation rates between wild-type (WT) SL12653 strain and a SL12653 strain deleted for the comX gene (ComX−); transformation rate of three clones of the ComX-deficient strain compared to the WT strain, in presence (+DNA) or in absence (−DNA) of donor DNA.

### DETAILED DESCRIPTION

The present invention is based on the observation that overexpression of ComX in a strain of the Lactococcus genus allowed to transform this strain by natural competence. Using this approach a L. lactis strain was generated by natural transformation with an exogenous DNA polynucleotide. Importantly, these results are the first demonstration of transformation of a L. lactis strain by natural competence. Further, existence of natural competence in the Lactococcus genus has been confirmed in two strains of the Lactococcus raffinolactis species and two Lactococcus lactis species.

The practice of the present invention will employ, unless otherwise indicated, conventional techniques of molecular biology, biochemistry, microbiology, bacteriology, and related fields, which are within the capabilities of a person of ordinary skill in the art. Such techniques are explained in the literature.

Thus, the present invention provides a method for transforming a strain of the Lactococcus genus with an exogenous DNA polynucleotide comprising the steps of:
(a) providing a strain of the Lactococcus genus, wherein said strain is transformable through natural competence;
(b) modulating the production of a ComX protein in said strain;
(c) contacting said strain of step (b) with an exogenous DNA polynucleotide in a medium and incubating the resulting mixture for integration of the exogenous DNA polynucleotide into the genome of said strain; and
(d) selecting a strain which has integrated the exogenous DNA polynucleotide into its genome.

As detailed below, step (b) and step c) can be carried out sequentially [i.e., step (b) and then step (c)] or in another embodiment step (b) and step (c) can be carried out simultaneously.

Lactococcus Genus

The present invention relates to a method for transforming a strain of the Lactococcus genus, a Gram-positive bacterium. Lactococcus strains are known as lactic acid bacteria (LAB) for their ability to convert carbohydrate to lactic acid. A strain of the Lactococcus genus and Lactococcus strain are used herein interchangeably.

The Lactococcus genus comprises, but is not limited to the following species: Lactococcus chungangensis, Lactococcus fujiensis, Lactococcus garvieae, Lactococcus lactis, Lactococcus piscium, Lactococcus plantarum and Lactococcus raffinolactis. Any strain of one of these species may be used in the current invention, provided that this strain is transformable through natural competence as defined herein.

In a particular embodiment, said strain of the Lactococcus genus of step a) is a strain of the Lactococcus lactis species or a strain of the Lactococcus raffinolactis species.

Lactococcus lactis

In a particular embodiment, said strain of the Lactococcus genus of step a) is a strain of the Lactococcus lactis species. The species Lactococcus lactis comprises several subspecies. Thus, when the strain of the Lactococcus genus of step a) is a strain of the Lactococcus lactis species, said strain is selected in the group consisting of Lactococcus lactis subsp. cremoris, Lactococcus lactis subsp. hordniae, Lactococcus lactis subsp. lactis and Lactococcus lactis subsp. tructae. As used herein a strain of the Lactococcus lactis species is understood to be a genetic variant or subtype of any L. lactis species or subspecies. The different Lactococcus lactis subspecies disclosed here, and in particular the lactis and the cremoris subspecies, are defined herein based on DNA sequences coding for 16S ribosomal RNA [Ward et al., 1998].

In a particular aspect, the present invention provides a method for transforming a strain of the Lactococcus lactis species with an exogenous DNA polynucleotide comprising the steps of:
(a) providing a strain of the Lactococcus lactis species, wherein said strain is transformable through natural competence;
(b) modulating the production of a ComX protein in said strain;
(c) contacting said strain of step (b) with an exogenous DNA polynucleotide in a medium and incubating the resulting mixture for integration of the exogenous DNA polynucleotide into the genome of said strain; and
(d) selecting a strain which has integrated the exogenous DNA polynucleotide into its genome.

In a preferred embodiment, the strain of step (a) is a Lactococcus lactis subsp. cremoris strain or a Lactococcus lactis subsp. lactis strain. Both subspecies have been identified and characterised with full genome sequences-see, e.g., Wegmann et al. (2007) J. Bacteriol. 189:3256-3270 and Bolotin et al. (2001) Genome Res. 11:731-753. With regards to the dairy industry, L. lactis subsp. lactis (previously known as Streptococcus lactis) is preferred for making soft cheese while L. lactis subsp. cremoris (previously known as Streptococcus cremoris) is preferred for hard cheese production.

In a preferred embodiment, the strain of step (a) is Lactococcus lactis subsp. cremoris strain. In another preferred embodiment, the strain of step (a) is Lactococcus lactis subsp. lactis strain.

Lactococcus raffinolactis

In a particular embodiment, said strain of the Lactococcus genus of step a) is a strain of the Lactococcus raffinolactis species.

In a particular aspect, the present invention provides a method for transforming a strain of the Lactococcus raffinolactis species with an exogenous DNA polynucleotide comprising the steps of:
(a) providing a strain of the Lactococcus raffinolactis species, wherein said strain is transformable through natural competence;
(b) modulating the production of a ComX protein in said strain;
(c) contacting said strain of step (b) with an exogenous DNA polynucleotide in a medium and incubating the resulting mixture for integration of the exogenous DNA polynucleotide into the genome of said strain; and
(d) selecting a strain which has integrated the exogenous DNA polynucleotide into its genome.

Lactococcus plantarum

In a particular embodiment, said strain of the Lactococcus genus of step a) is a strain of the Lactococcus plantarum species.

In a particular aspect, the present invention provides a method for transforming a strain of the Lactococcus plantarum species with an exogenous DNA polynucleotide comprising the steps of:
(a) providing a strain of the Lactococcus plantarum species, wherein said strain is transformable through natural competence;
(b) modulating the production of a ComX protein in said strain;
(c) contacting said strain of step (b) with an exogenous DNA polynucleotide in a medium and incubating the resulting mixture for integration of the exogenous DNA polynucleotide into the genome of said strain; and
(d) selecting a strain which has integrated the exogenous DNA polynucleotide into its genome.
   Lactococcus piscium

In a particular embodiment, said strain of the Lactococcus genus of step a) is a strain of the Lactococcus piscium species.

In a particular aspect, the present invention provides a method for transforming a strain of the Lactococcus piscium species with an exogenous DNA polynucleotide comprising the steps of:
(a) providing a strain of the Lactococcus piscium species, wherein said strain is transformable through natural competence;
(b) modulating the production of a ComX protein in said strain;
(c) contacting said strain of step (b) with an exogenous DNA polynucleotide in a medium and incubating the resulting mixture for integration of the exogenous DNA polynucleotide into the genome of said strain; and
(d) selecting a strain which has integrated the exogenous DNA polynucleotide into its genome.
   Lactococcus garvieae

In a particular embodiment, said strain of the Lactococcus genus of step a) is a strain of the Lactococcus garvieae species.

In a particular aspect, the present invention provides a method for transforming a strain of the Lactococcus garvieae species with an exogenous DNA polynucleotide comprising the steps of:
(a) providing a strain of the Lactococcus garvieae species, wherein said strain is transformable through natural competence;
(b) modulating the production of a ComX protein in said strain;
(c) contacting said strain of step (b) with an exogenous DNA polynucleotide in a medium and incubating the resulting mixture for integration of the exogenous DNA polynucleotide into the genome of said strain; and
(d) selecting a strain which has integrated the exogenous DNA polynucleotide into its genome.
   Lactococcus fujiensis

In a particular embodiment, said strain of the Lactococcus genus of step a) is a strain of the Lactococcus fujiensis species.

In a particular aspect, the present invention provides a method for transforming a strain of the Lactococcus fujiensis species with an exogenous DNA polynucleotide comprising the steps of:
(a) providing a strain of the Lactococcus fujiensis species, wherein said strain is transformable through natural competence;
(b) modulating the production of a ComX protein in said strain;
(c) contacting said strain of step (b) with an exogenous DNA polynucleotide in a medium and incubating the resulting mixture for integration of the exogenous DNA polynucleotide into the genome of said strain; and
(d) selecting a strain which has integrated the exogenous DNA polynucleotide into its genome.
   Lactococcus chungangensis

In a particular embodiment, said strain of the Lactococcus genus of step a) is a strain of the Lactococcus chungangensis species.

In a particular aspect, the present invention provides a method for transforming a strain of the Lactococcus chunganensis species with an exogenous DNA polynucleotide comprising the steps of:
(a) providing a strain of the Lactococcus chungangensis species, wherein said strain is transformable through natural competence;
(b) modulating the production of a ComX protein in said strain;
(c) contacting said strain of step (b) with an exogenous DNA polynucleotide in a medium and incubating the resulting mixture for integration of the exogenous DNA polynucleotide into the genome of said strain; and
(d) selecting a strain which has integrated the exogenous DNA polynucleotide into its genome.

### DNA Acquisition

Bacteria may naturally acquire exogenous DNA via one of three possible mechanisms: transformation, conjugation, or transduction.

As used herein the term “transformation” refers to the uptake of exogenous genetic material (e.g. a DNA polynucleotide) from the external medium. Since transformation requires that genetic material cross the bacterial cell wall and membrane and the uptake of exogenous genetic material is energetically costly, the process is tightly regulated. Accordingly, bacterial cells may only be transformed under certain conditions. Bacterial cells which are in a transformable state are said to be competent.

Competence may be artificially induced in the laboratory, e.g. by electroporation or exposure to divalent cations (e.g. CaCl₂)) and heat shock. Alternatively, some species of bacteria express a proteinaceous machinery that provides natural competence; this system of natural competence has been widely studied in streptococci.

As used herein the term “conjugation” refers to the transfer of genetic material between bacterial cells.

As used herein the term “transduction” refers to the transfer of genetic material from a virus (e.g. a bacteriophage) or a viral vector into bacterial cell.

### ComX Protein

The method of the present invention comprises the step of modulating the production of a ComX protein in said strain.

ComX protein is an alternative sigma factor, also known as ox, which acts as master regulator for the late com genes and is responsible for transcriptional reprogramming of cells including the induction of genes strictly required for DNA transformation (Lee et al., 1989; Petersen et al. 2004).

ComX may bind to a specific target sequence (or box) termed the Com-box (or Cin-box). Com-boxes are located in the vicinity of the promoters of late competence (com) genes/operons responsible for DNA uptake (e.g., comG, comF, and comE operons), DNA protection (e.g. ssb) and DNA recombination (e.g. recA, dprA, coiA), and positively controls their expression (Campbell et al., 1998; Luo and Morrisoin, 2003).

The production of the ComX protein in a strain of interest may be increased relatively to an appropriate control strain, i.e., the Lactococcus strain in which the production of the ComX protein has not been modulated. ComX protein may be produced (expressed) following modulation as compared to an appropriate control strain, i.e., the Lactococcus strain in which the ComX protein is not produced.

In some embodiments, the production of the ComX protein is constitutive or inducible.

The production of ComX protein may be monitored using any method known in the art. For example, by western blotting using an antibody specific for the ComX protein. Alternatively, comX gene mRNA transcript levels may be measured by qPCR.

Alternatively, the ComX protein may be monitored using a reporter construct polynucleotide, e.g. as described in the Example 1 and Materials and Methods. The reporter construct polynucleotide may comprise genes encoding one or more reporter proteins, preferably the genes encoding the reporter proteins are operably linked to a promoter comprising a Com-box sequence. The reporter proteins may be LuxAB or Luc. Accordingly, ComX expression (and activity) may be detected and measured using a luciferase assay (Fontaine et al., 2010).

In some embodiments of the method of the present invention, the step of modulating the production of a ComX protein is performed by expressing a comX gene in said strain or increasing the expression of a comX gene in said strain. In a particular embodiment, the step of modulating the production of a ComX protein is performed by expressing a comX gene in said strain in some growth conditions, whereas said strain does not express the ComX protein outside of these growth conditions. In a particular embodiment, the step of modulating the production of a ComX protein is performed by increasing the expression of a comX gene in said strain in some growth conditions.

The comX gene may be an exogenous comX gene. As used herein an “exogenous comX gene” is understood to be a comX gene which is brought into the cytoplasm of the Lactococcus strain of step a), in order to be expressed. The exogenous comX gene may have the same sequence as the comX gene found in the genome of the Lactococcus strain of step a) or may have a different sequence from the comX gene found in the genome of the Lactococcus strain of step a). When different, the comX gene may be derived from a strain of a different species, a different subspecies or a different strain of Lactococcus.

The exogenous comX gene may be integrated within the genome of said Lactococcus strain. Alternatively, the exogenous comX gene may be located within a vector. The vector may be selected from a plasmid, a viral vector (e.g. a phage), a cosmid, or a bacterial artificial chromosome.

Said plasmid may be transferred into said Lactococcus strain by conjugation, transformation or transduction. Said plasmid may be auto-replicative in the transformed Lactococcus strain or not.

The exogenous comX gene may be operably linked to transcription regulator(s). The exogenous comX gene may be located in a linear or circular polynucleotide.

Alternatively, in some embodiments of the method of the present invention, the comX gene is the endogenous comX gene of said Lactococcus strain. As used herein “the endogenous comX gene of said strain” is understood to be a comX gene that is naturally present in the genome of said strain.

In some embodiments, said comX gene is a Lactococcus comX gene. In an embodiment, said comX gene is a Lactococcus lactis comX gene. In a particular embodiment, said comX gene is a Lactococcus lactis subsp. lactis comX gene. In a particular embodiment, said comX gene is a Lactococcus lactis subsp. cremoris comX gene.

The comX gene may comprise or consist of a nucleotide sequence selected from the group consisting of:
SEQ ID NO:1, SEQ ID NO:3, SEQ ID NO:5, SEQ ID NO:7, SEQ ID NO:9, SEQ ID NO: 11, SEQ ID NO: 13, SEQ ID NO:15, SEQ ID NO:17, SEQ ID NO: 19; SEQ ID NO:21;
a nucleotide sequence having at least 90%, at least 91%, at least 92%, at least 93%, at least 94%, at least 95%, at least 96%, at least 97%, at least 98%, or at least 99% identity to the nucleotide sequence of SEQ ID NO:1, SEQ ID NO:3, SEQ ID NO:5, SEQ ID NO: 7, SEQ ID NO:9, SEQ ID NO:11, SEQ ID NO:13, SEQ ID NO: 15, SEQ ID NO:17, SEQ ID NO:19; SEQ ID NO:21;
a variant of SEQ ID NO:1, SEQ ID NO:3, SEQ ID NO:5, SEQ ID NO:7, SEQ ID NO:9, SEQ ID NO:11, SEQ ID NO: 13, SEQ ID NO:15, SEQ ID NO: 17, SEQ ID NO: 19, SEQ ID NO: 21 encoding respectively a ComX protein of SEQ ID NO:2, SEQ ID NO:4, SEQ ID NO: 6, SEQ ID NO:8, SEQ ID NO:10, SEQ ID NO:12, SEQ ID NO:14, SEQ ID NO:16, SEQ ID NO:18, SEQ ID NO:20 or SEQ ID NO:22; and
a variant of SEQ ID NO:1, SEQ ID NO:3, SEQ ID NO:5, SEQ ID NO:7, SEQ ID NO:9, SEQ ID NO:11, SEQ ID NO: 13, SEQ ID NO: 15, SEQ ID NO:17, SEQ ID NO:19, SEQ ID NO: 21 encoding respectively a functional ComX protein having at least 90% identity or at least 90% similarity to a ComX protein of SEQ ID NO:2, SEQ ID NO:4, SEQ ID NO:6, SEQ ID NO:8, SEQ ID NO:10, SEQ ID NO:12, SEQ ID NO:14, SEQ ID NO: 16, SEQ ID NO: 18, SEQ ID NO:20 or SEQ ID NO:22.

[SEQ ID NO: 1]
ATAACATATTACTTGGAAGAAGAGGATTTTGAAAATCTTTTTTCAGAAATGAAACCTATAGTTATGAA
ATTAATGAAACAAATTCGCATTAGAACATGGAAAATAGAGGATTATCTTCAAGAGGGGATGATTATTT
TACATCTTCTATTAGAAGAGCAGAACGATGGTCAAAAGCTGCATACAAAATTTAAGGTAAAGTATCAT
CAAAGATTAATAGATGAATTAAGACGAAGTTATGCAAAGAAACGAAGCCATGACCATTTTATAGGTTT
AGATGTTTATGAATGCTCAGACTGGATAAATTCAGGTGATACTAGTCCAGATAATGAAGTGGTCTTCA
ATCATTTGCTGGCAGAAGTATATGAAGGTTTGAGCGCACATTATCAAGACTTACTACTTCGACAAATG
CGAGGAGAAGAACTAACTCGCATGCAACGGTATCGCCTTCGTGAAAAAATAAAGGCCATCTTATTTTC
AGAAGACGAAGAGTGA
[SEQ ID NO: 2]
MTYYLEEEDFENLFSEMKPIVMKLMKQIRIRTWKIEDYLQEGMIILHLLLEEQNDGQKLHTKFKVKYH
QRLIDELRRSYAKKRSHDHFIGLDVYECSDWINSGDTSPDNEVVFNHLLAEVYEGLSAHYQDLLLRQM
RGEELTRMQRYRLREKIKAILFSEDEE
[SEQ ID NO: 3]
ATGACATATTACCTGGAAGAAAATGAATTCGAAGGTTTATTTTCTGGAATGAAACCAATCATCAGAAA
ATTGATGAAACAAATTCGAATCAAAGCATGGGACATAGAGGATTATTATCAAGAAGGAATGATTATTT
TGCATCACCTTTTAGAAGAAAATCACCCATCCACTAATATTTATACAAAGTTCAAAGTAAAATATCAT
CAACATTTGATTGATGAACTACGCCATAGCTACGCCAAAAAACGGCTTCATGACCATTTTGTAGGTCT
GGACATTTATGAATGTTCGGACTGGATAGATGCAGGAGGAAGTACCCCTGAAAGCGAGCTTGTGTTCA
ATCATCTTTTAGCAGAAGTTTATGAAGGATTGAGCGCCCACTATCAGGAATTACTCGTGCGTCAAATG
AGAGGAGAAGAACTCACGCGAATGGAACGCTATCGGCTAAGAGAAAAAATCAAAAATATACTATTTTC
TCGAGATGATGATTAA
[SEQ ID NO: 4]
MTYYLEENEFEGLFSGMKPIIRKLMKQIRIKAWDIEDYYQEGMIILHHLLEENHPSTNIYTKFKVKYH
QHLIDELRHSYAKKRLHDHFVGLDIYECSDWIDAGGSTPESELVFNHLLAEVYEGLSAHYQELLVRQM
RGEELTRMERYRLREKIKNILFSRDDD
[SEQ ID NO: 5]
ATGGATGACATTCAAGAAAAATACGGTTTAGAATTCAACGAATTATTCTCTGAGATGCGGCCGATAAT
TTATAAATTGATGAAGCAATTGCACATCAACACATGGGATTACGATGATTACTTCCAAGAGGGAATGA
TTACACTACATGAATTGCTGCAGAAAATTACAAATTTAGATCATGTACATACGAAATTTAAAGTGGCT
TACCATCAGCACTTAATTGACGAAATTCGCCATATTAAAGCACGAAAAAGAGGTTTTGATCAGCTCCA
TCCGATCAATGTTTATGACTGCGCAGATTGGATTGGCTCAAACCTTGCTACACCTGAAAGCGAGATAG
TTTTCAACCATCTACTAGAAGAAGTTTATGATAAACTTTCAACACACTATAAAGAACTGTTGGTAAAG
CAAATGCATGGGGAACATCTTACGAGAATGCAGAAGTATCGTTTAAAGGAAAAAATTAAAGCGATTTT
ATTTGATGAAGACTAA
[SEQ ID NO: 6]
MDDIQEKYGLEFNELFSEMRPIIYKLMKQLHINTWDYDDYFQEGMITLHELLQKITNLDHVHTKFKVA
YHQHLIDEIRHIKARKRGFDQLHPINVYDCADWIGSNLATPESEIVFNHLLEEVYDKLSTHYKELLVK
QMHGEHLTRMQKYRLKEKIKAILFDED
[SEQ ID NO: 7]
ATGGATAAAATTGAAACCATACTTAAAAGTATTGAACCGATTATTATGAACTGTCGGAAAAAAACTAA
AATTCCTTCCTGGGAATTAGACGACTATATGCAGGAAGGGATGATTATTGCTTTAGAGATGTACCATC
AACTCTTATTAGATCCACCAGATGATGACTTTAACTTCTATGTCTATTTCAAAGTCAGGTATTCTTGT
TTCTTAATTGATCACTATCGCAAAGCTATGGCAGTCAAGAGAAAATTCGACCAGCTTGACTATTGTGA
ACTTTCTGAGTCTGTTAATCTTTTTGATCACAAACAAAATGTGTCTGAAAACGTCATGTATAACTTGT
TGTGTCAAGAAATACACTTGGTTTTATCCCCGGAGGAGCTCAAGCTTTTTGAGGCACTTATTTGA
[SEQ ID NO: 8]
MDKIETILKSIEPIIMNCRKKTKIPSWELDDYMQEGMIIALEMYHQLLLDPPDDDFNFYVYFKVRYSC
FLIDHYRKAMAVKRKFDQLDYCELSESVNLFDHKQNVSENVMYNLLCQEIHLVLSPEELKLFEALI
[SEQ ID NO: 9]
ATGGATAGCATAGAAATGATGCTTCAAAATATTGAGCCAATTATTATGAATTGTAGTAAAACAACTAG
GATTCCATCTTGGGAGCTAGATGATTACATGCAGGAGGGGATGATTATTGCACTGGAAATGTATCAAA
ATAGACATAACATCAATAACGGTAACGCGTTTAATTTCTATGTCTATTTTAAAGTCAGGTATTCCTGT
TACCTGATAGATAGTTTTAGAAAGGCTAACGCATATAAAAGAAAATTTGATCAACCATTATATTGTGA
AATATCTGAAGCCTTCAACCTTTATGATCACCACCAAAATGTTGCAGACAATGTCTGTTATCAGCTAT
TGCAAGTTGAAATTCTTGAGATATTAACACCAGATGAAGCTGATTTATTTATGACCTTGAAAAATGGT
GGGAAAGTAGAGAGAAATAAAAAGTATAGATTAAAGAAAAAAATTATTGATTATCTTAAAGACATGTT
ATGA
[SEQ ID NO: 10]
MDSIEMMLQNIEPIIMNCSKTTRIPSWELDDYMQEGMIIALEMYQNRHNINNGNAFNFYVYFKVRYSC
YLIDSFRKANAYKRKFDQPLYCEISEAFNLYDHHQNVADNVCYQLLQVEILEILTPDEADLFMTLKNG
GKVERNKKYRLKKKIIDYLKDML
[SEQ ID NO: 11]
ATGGAGACTTTAGAAGCCATGCTCAAAAACATTGAACCTATTATTATGAATTGTCAAAAGATGGCAAA
AATACCTTCCTGGGATATTGACGATTATATGCAGGAGGGGAGGATCATTGCATTAGACTTGTATAATC
AGCTAGCAGAAAGAATGGAGACGGATGAGGTGAACTTTTACGTCTACTTCAAAGTCAGATATACCTGT
TTCTTGATTGATACTTACCGTAAGACAAATGCCTTTAAAAGAAAATTTGACCAACCGATTTACTTAGA
TGTATCCGAAGCATTTAATCTGTATGATCATAAGCAGAATGTCGCTGATAATGTCATGTATACTTTAT
TGCATCAGGAGATTCTAGACATCTTAACGCCTGTAGAAATTCAAACGCTAAACGCACTAAAAAGGGGA
GAAAAGGTCGACCGCAATAAAAAATTTAGGATTAAAAAGAAGATTATCAACTATATTAATCAGATTTT
CTAG
[SEQ ID NO: 12]
METLEAMLKNIEPIIMNCQKMAKIPSWDIDDYMQEGRIIALDLYNQLAERMETDEVNFYVYFKVRYTC
FLIDTYRKTNAFKRKFDQPIYLDVSEAFNLYDHKQNVADNVMYTLLHQEILDILTPVEIQTLNALKRG
EKVDRNKKFRIKKKIINYINQIF
[SEQ ID NO: 13]
ATGGAGCATAATTTAGATATGGAGCAGCTGGAAGAAATTTTTCATTCTGTCCAACATATTGTGTGGAA
GAACAGTCGTTTGATTCCGATAAATTTTTGGACGTTTGATGACTATCAGCAGGAAGGGCGCTTGGTAT
TATACGATTTGCTGGGAGATGGTGTGACGCAAAGGAACTTATTTTGCCATTTTAAGGTACGCTATAAG
CAGAGACTTATTGATATTAAAAGAAGGGAGCGGGCTTTTAAAAGGGGTTTTGATTGCGGGACTGGCTT
AGATATATACGAATATTCTGATGCTCTAAAGGGGAAAGCAGCCAGTCCAGAACATATCCTGATTTCTG
GAAGTTTACTTGAAGAAGTTTTTGAAAACTTAAATTTACGCTACCGACGGCTCCTCAAAAGTTACCTC
GCCGGCGATGAATTGCACCGTATGGAAAAGTATCGTTTGAAGGAAAAAATAACGAATATATTATATGA
ACAGCAGTGA
[SEQ ID NO 14]
MEHNLDMEQLEEIFHSVQHIVWKNSRLIPINFWTFDDYQQEGRLVLYDLLGDGVTQRNLFCHFKVRYK
QRLIDIKRRERAFKRGFDCGTGLDIYEYSDALKGKAASPEHILISGSLLEEVFENLNLRYRRLLKSYL
AGDELHRMEKYRLKEKITNILYEQQ
[SEQ ID NO: 15]
ATGGCAGAAAATAATTTAGATAAAGAACAGCTTGAAGAGTTATTCCATTCACTTCAACATATTGTTTG
GAAGAACAGTCATTTAATTAAAATAAATTTTTGGACAATGGATGATTATCAGCAAGAAGGGCGACTGG
TTTTATACCAGTTACTTGAAGATGGCGTGACACAGGAAAAACTATTTTGCCATTTTAAAGTGCGATAT
AAGCAACGGTTGATTGATATAAAAAGACGAGAAAGAGCATTTAAGCGGGGTTTTGATTGTGGGGCTGG
TTTAGATATATATGAGTATTCTGATGCCCTGAAAGGCAAAGCTACCAGTCCTGAATATAACTTAATTT
CAGTTACTTTACTTGAAGAGGTTCATCAAAGTTTGAGTTTGAGATACCGCAATTTATTGGAGAATCAT
CTGTCAGGAGTGGAGTTGCATCGAATGGAAAAATACCGTTTAAAGGAAAAAATCAAGAGAATACTCTA
TGAAGAAGAATGA
[SEQ ID NO: 16]
MAENNLDKEQLEELFHSLQHIVWKNSHLIKINFWTMDDYQQEGRLVLYQLLEDGVTQEKLFCHFKVRY
KORLIDIKRRERAFKRGFDCGAGLDIYEYSDALKGKATSPEYNLISVTLLEEVHQSLSLRYRNLLENH
LSGVELHRMEKYRLKEKIKRILYEEE
[SEQ ID NO: 17]
ATGGAGCATAATTTAGATATGGAGCAGCTGGAAGAGATATTTCATTCTGTTCAACATATTGTATGGAA
GAATAGTCGTTTGATTCCGATAAATTTTTGGACGATAGATGACTATCAGCAGGAAGGGCGTTTGGTAT
TATATGATTTACTTGAGGATGGTGTGACACAAAGAAAACTTTTTTGCCATTTTAAAGTACGTTATAAG
CAGAGACTTATTGATATTAAAAGAAGGGAGCGGGCTTTTAAAAGGGGTTTTGACTGTGGGACTGGGCT
AGATATTTACGAATATTCAGATGCTTTAAAAGGAAAAGTAGCCAGTCCAGAACATACTCTGATTTCTG
GCAGTTTGCTTGAAGAAGTTTTAGAAAACTTAAATTTACGCTACCGTGCTCTTCTTAAAAGTTACCTT
GCTGGTGATGAACTGCATCGAATGGAAAAACATCGTTTGAAAGAAAAAATAATAAAAATATTATATGA
TGAACAGTGA
[SEQ ID NO: 18]
MEHNLDMEQLEEIFHSVQHIVWKNSRLIPINFWTIDDYQQEGRLVLYDLLEDGVTQRKLFCHFKVRYK
QRLIDIKRRERAFKRGFDCGTGLDIYEYSDALKGKVASPEHTLISGSLLEEVLENLNLRYRALLKSYL
AGDELHRMEKHRLKEKIIKILYDEQ
[SEQ ID NO: 19]
TTGAAACCGATCGTTTCAAAATCTATGAGAACATTAAAAATCAATTTTTGGACTACAGAGGATTATCA
TCAAGAGGGTCTAATTACATTAAATGAAATATTAAATTCAGGATGTAAGGAGTCACAACTATACATTC
ACTTTAAAGTCAAATATCGACAAAAGCTAATAGACGTGATTAGAAAATCACAGGCGCAAAAAAGAATC
TGGGATAATGCAGAGAGTATTGATGTTTACGAATCTGAAAATCAAATTAATTCCAGTAACTCAAACCC
CGAAGACATAATAGTCTATGACAGTCTTGTAAAGGAAGTAATAACAAAATTAACACCTTCATACCGGA
AACTACTGAAACGACATCTAAGAGGTGAGGATGTGACAAGGATGGAAAAATACAGACTGAAGGAACGA
ATCAAACAAATTTTATTTGATGGTGATTGA
[SEQ ID NO: 20]
MKPIVSKSMRTLKINFWTTEDYHQEGLITLNEILNSGCKESQLYIHFKVKYRQKLIDVIRKSQAQKRI
WDNAESIDVYESENQINSSNSNPEDIIVYDSLVKEVITKLTPSYRKLLKRHLRGEDVTRMEKYRLKER
IKQILFDGD
[SEQ ID NO: 21]
ATGGATAAGATTGAAACCATACTTAAAAATATTGAACCGATTATCATGAACTGTCGAAAAAAAACTAA
CATCCCTTCCTGGCAATTAGACGACTATCTCCAGGAAGGCATGATTATTGCTCTAGAGATGTATCATC
AACTTTTATTAGACCCACCAGATGATGACTTTAACTTCTATGTTTATTICAAAGTGAGATATTCTTGT
TTCTTGATTGATCAGTATCGGAGAAACATGGCTGTCAAAAGAAAATTCGACCAGATTGACTATTGTGA
ACTATCTGAGGCGTTTTATCTTTTTGATCAAAATCAAGATGTCTCTGAAAACGTCATGTATAATTTGT
TATGTCAAGAAATACACTTGCTTCTATCTCCTGAAGAACGAGAGCTTTTTGAGGCACTTAAAAATGGA
CAGAAGATTGACCGTAATCAAAAGTTTCGTATCAAGAAGAAAATTATTGAATATATTAAGAGGTTTTG
GTGA
[SEQ ID NO: 22]
MDKIETILKNIEPIIMNCRKKTNIPSWQLDDYLQEGMIIALEMYHQLLLDPPDDDFNFYVYFKVRYSC
FLIDQYRRNMAVKRKFDQIDYCELSEAFYLFDQNQDVSENVMYNLLCQEIHLLLSPEERELFEALKNG
QKIDRNOKFRIKKKIIEYIKREW

In some embodiments, said comX gene has the nucleotide sequence of SEQ ID NO:1 or SEQ ID NO: 3 or SEQ ID NO:5 or has at least 90%, at least 91%, at least 92%, at least 93%, at least 94%, at least 95%, at least 96%, at least 97%, at least 98%, or at least 99% identity to the nucleotide sequence of SEQ ID NO:1 or SEQ ID NO:3 or SEQ ID NO:5 or is a variant of SEQ ID NO: 1 or SEQ ID NO:3 or SEQ ID NO:5 encoding respectively the ComX protein of SEQ ID NO: 2, SEQ ID NO:4 or SEQ ID NO:6 or is a variant of SEQ ID NO:1 or SEQ ID NO:3 or SEQ ID NO: 5 encoding respectively a functional ComX protein having at least 90% identity or at least 90% similarity to a ComX protein of SEQ ID NO:2, SEQ ID NO:4 or SEQ ID NO:6. In a particular embodiment, said comX gene is used when the Lactococcus strain in step a) is a Lactococcus lactis strain.

In a particular embodiment, when the strain of step a) is a Lactococcus lactis subsp. lactis strain, the comX gene comprises the nucleotide sequence of SEQ ID NO:1, any sequence having at least 90%, at least 91%, at least 92%, at least 93%, at least 94%, at least 95%, at least 96%, at least 97%, at least 98%, or at least 99% identity to SEQ ID NO:1, a variant of SEQ ID NO:1 encoding the ComX protein of SEQ ID NO:2 or a variant of SEQ ID NO:1 encoding a functional ComX protein having at least 90% identity or at least 90% similarity to a ComX protein of SEQ ID NO:2.

In a particular embodiment, when the strain of step a) is a Lactococcus lactis subsp. cremoris strain, the comX gene comprises the nucleotide sequence of SEQ ID NO:3 or SEQ ID NO:5, any sequence having at least 90%, at least 91%, at least 92%, at least 93%, at least 94%, at least 95%, at least 96%, at least 97%, at least 98%, or at least 99% identity to SEQ ID NO:3 or SEQ ID NO:5 or a variant of SEQ ID NO:3 or SEQ ID NO:5 encoding respectively the ComX protein of SEQ ID NO:4 or SEQ ID NO:6 or a variant of SEQ ID NO:3 or SEQ ID NO:5 encoding respectively a functional ComX protein having at least 90% identity or at least 90% similarity to a ComX protein of SEQ ID NO:4 or SEQ ID NO:6.

In some embodiments, said comX gene has the nucleotide sequence of SEQ ID NO:7 or has at least 90%, at least 91%, at least 92%, at least 93%, at least 94%, at least 95%, at least 96%, at least 97%, at least 98%, or at least 99% identity to the nucleotide sequence of SEQ ID NO: 7 or is a variant of SEQ ID NO:7 encoding the ComX protein of SEQ ID NO:8, or is a variant of SEQ ID NO:7 encoding a functional ComX protein having at least 90% identity or at least 90% similarity to a ComX protein of SEQ ID NO:8. In a particular embodiment, said comX gene is used when the Lactococcus strain in step a) is a Lactococcus raffinolactis strain.

In some embodiments, said comX gene has the nucleotide sequence of SEQ ID NO:9 or has at least 90%, at least 91%, at least 92%, at least 93%, at least 94%, at least 95%, at least 96%, at least 97%, at least 98%, or at least 99% identity to the nucleotide sequence of SEQ ID NO: 9 or is a variant of SEQ ID NO:9 encoding the ComX protein of SEQ ID NO:10, or is a variant of SEQ ID NO:9 encoding a functional ComX protein having at least 90% identity or at least 90% similarity to a ComX protein of SEQ ID NO:10. In a particular embodiment, said comX gene is used when the Lactococcus strain in step a) is a Lactococcus plantarum strain.

In some embodiments, said comX gene has the nucleotide sequence of SEQ ID NO:11 or has at least 90%, at least 91%, at least 92%, at least 93%, at least 94%, at least 95%, at least 96%, at least 97%, at least 98%, or at least 99% identity to the nucleotide sequence of SEQ ID NO: 11 or is a variant of SEQ ID NO:11 encoding the ComX protein of SEQ ID NO:12, or is a variant of SEQ ID NO:11 encoding a functional ComX protein having at least 90% identity or at least 90% similarity to a ComX protein of SEQ ID NO:12. In a particular embodiment, said comX gene is used when the Lactococcus strain in step a) is a Lactococcus piscium strain.

In a particular embodiment, said comX gene has the nucleotide sequence of SEQ ID NO:13 or SEQ ID NO:15 or SEQ ID NO:17, any sequence having at least 90%, at least 91%, at least 92%, at least 93%, at least 94%, at least 95%, at least 96%, at least 97%, at least 98%, or at least 99% identity to SEQ ID NO:13 or SEQ ID NO:15 or SEQ ID NO:17 or a variant of SEQ ID NO: 13 or SEQ ID NO:15 or SEQ ID NO:17 encoding respectively the ComX protein of SEQ ID NO: 14 or SEQ ID NO:16 or SEQ ID NO:18 or a variant of SEQ ID NO:13 or SEQ ID NO:15 or SEQ ID NO: 17 encoding respectively a functional ComX protein having at least 90% identity or at least 90% similarity to a ComX protein of SEQ ID NO:14 or SEQ ID NO:16 or SEQ ID NO: 18. In a particular embodiment, said comX gene is used when the Lactococcus strain in step a) is a Lactococcus garvieae strain.

In some embodiments, said comX gene has the nucleotide sequence of SEQ ID NO:19 or has at least 90%, at least 91%, at least 92%, at least 93%, at least 94%, at least 95%, at least 96%, at least 97%, at least 98%, or at least 99% identity to the nucleotide sequence of SEQ ID NO: 19 or is a variant of SEQ ID NO:19 encoding the ComX protein of SEQ ID NO:20, or is a variant of SEQ ID NO:19 encoding a functional ComX protein having at least 90% identity or at least 90% similarity to a ComX protein of SEQ ID NO:20. In a particular embodiment, said comX gene is used when the Lactococcus strain in step a) is a Lactococcus fujiensis strain.

In some embodiments, said comX gene has the nucleotide sequence of SEQ ID NO:21 or has at least 90%, at least 91%, at least 92%, at least 93%, at least 94%, at least 95%, at least 96%, at least 97%, at least 98%, or at least 99% identity to the nucleotide sequence of SEQ ID NO: 21 or is a variant of SEQ ID NO:21 encoding the ComX protein of SEQ ID NO:22, or is a variant of SEQ ID NO:21 encoding a functional ComX protein having at least 90% identity or at least 90% similarity to a ComX protein of SEQ ID NO:22. In a particular embodiment, said comX gene is used when the Lactococcus strain in step a) is a Lactococcus chungangensis strain.

By way of example and for the avoidance of doubt, in particular embodiments, where a comX gene is specified as having a particular nucleotide sequence, it is understood that the comX gene comprises said nucleotide sequence. In particular other embodiments, where a comX gene is specified as having a particular nucleotide sequence, it is understood that the comX gene consists of said nucleotide sequence.

In some embodiments, variants as defined herein of comX genes are selected from the list of DNA sequences disclosed in Table 1 below:

TABLE 1

| Strain | Accession | Position of comX, from |
|---|---|---|
| Lactococcus lactis Al06 | CP009472.1 | From 2260881 to 2261372 |
| Lactococcus lactis Bpl1 | JRFX01000055.1 | From 34668 to 35159 |
| Lactococcus lactis Ll1596 | LDEK01000015.1 | From 33401 to 33892 |
| Lactococcus lactis subsp. cremoris A17 | JQIC01000009.1 | From 6445 to 6936 |
| Lactococcus lactis subsp. cremoris A76 | CP003132.1 | From 2293232 to 2293723 |
| Lactococcus lactis subsp. cremoris AM2 | LITE01000081.1 | From 9954 to 10444 |
| Lactococcus lactis subsp. cremoris B40 | LITC01000320.1 | From 10186 to 10677 |
| Lactococcus lactis subsp. cremoris | LAVW01000168.1 | From 445 to 936 (reverse) |
| Lactococcus lactis subsp. cremoris | AZSI01000020.1 | From 186 to 677 (reverse) |
| Lactococcus lactis subsp. cremoris HP | JAUH01000192.1 | From 40 to 531 (reverse) |
| Lactococcus lactis subsp. cremoris | JMMZ01000035.1 | From 92323 to 92814 |
| Lactococcus lactis subsp. cremoris | LIYF01000023.1 | From 40421 to 40912 |
| Lactococcus lactis subsp. cremoris KW2 | CP004884.1 | From 2276371 to 2276862 |
| Lactococcus lactis subsp. cremoris | LISZ01000238.1 | From 10034 to 10525 |
| Lactococcus lactis subsp. cremoris | JZUI01000076.1 | From 310 to 801 (reverse) |
| Lactococcus lactis subsp. cremoris | AM406671.1 | From 2376782 to 2377273 |
| Lactococcus lactis subsp. cremoris | BCVK01000073.1 | From 9879 to 10370 |
| Lactococcus lactis subsp. cremoris | CP002094.1 | From 2377598 to 2378089 |
| Lactococcus lactis subsp. cremoris | CP000425.1 | From 2283008 to 2283498 |
| Lactococcus lactis subsp. cremoris | ASXF01000005.1 | From 5621 to 6112 |
| Lactococcus lactis subsp. cremoris | ATBE01000400.1 | From 431 to 922 (reverse) |
| Lactococcus lactis subsp. cremoris | ATBC01000090.1 | From 315 to 809 (reverse) |
| Lactococcus lactis subsp. cremoris | ATBB01000278.1 | From 265 to 756 (forward) |
| Lactococcus lactis subsp. cremoris | ATBA01000081.1 | From 5620 to 6111 |
| Lactococcus lactis subsp. cremoris | CP003157.1 | From 2107522 to 2108013 |
| Lactococcus lactis subsp. cremoris V4 | LIYG01000005.1 | From 8625 to 9116 |
| Lactococcus lactis subsp. hordniae | BCVL01000030.1 | From 70 to 561 (reverse) |
| Lactococcus lactis subsp. lactis 1AA59 | AZQT01000035.1 | From 118 to 609 (reverse) |
| Lactococcus lactis subsp. lactis 511 | JNLP01000001.1 | From 1703029 to 1703520 |
| Lactococcus lactis subsp. lactis A12 | LT599049.1 | From 2415707 to 2416198 |
| Lactococcus lactis subsp. lactis ATCC | LKLC01000004.1 | From 32310 to 32801 |
| Lactococcus lactis subsp. lactis bv. | LIWD01000119.1 | From 147 to 638 (reverse) |
| Lactococcus lactis subsp. lactis CV56 | CP002365.1 | From 2213300 to 2213791 |
| Lactococcus lactis subsp. lactis | LAVD01000101.1 | From 544 to 1035 (reverse) |
| Lactococcus lactis subsp. lactis E34 | LKLD01000014.1 | From 197 to 688 (reverse) |
| Lactococcus lactis subsp. lactis II1403 | AE005176.1 | From 2223528 to 2224019 |
| Lactococcus lactis subsp. lactis IO-1 | AP012281.1 | From 2287126 to 2287617 |
| Lactococcus lactis subsp. lactis JCM | BBAP01000017.1 | From 34164 to 34656 |
| Lactococcus lactis subsp. lactis K231 | LKLE01000041.1 | From 32159 to 32650 |
| Lactococcus lactis subsp. lactis K337 | LKLF01000041.1 | From 34909 to 35400 |
| Lactococcus lactis subsp. lactis KF134 | LKLJ01000010.1 | From 34939 to 35430 |
| Lactococcus lactis subsp. lactis KF147 | CP001834.1 | From 2446402 to 2446893 |
| Lactococcus lactis subsp. lactis KF201 | LKLM01000024.1 | From 28747 to 29238 |
| Lactococcus lactis subsp. lactis KF24 | LKLH01000011.1 | From 34116 to 34607 |
| Lactococcus lactis subsp. lactis KF282 | LKLN01000033.1 | From 170 to 661 (reverse) |
| Lactococcus lactis subsp. lactis KLDS | CP006766.1 | From 2407603 to 2408094 |
| Lactococcus lactis subsp. lactis LMG | JQCM01000018.1 | From 37736 to 38227 |
| Lactococcus lactis subsp. lactis | LKLQ01000046.1 | From 38499 to 38993 |
| Lactococcus lactis subsp. lactis NCDO | CP009054.1 | From 2402923 to 2403414 |
| Lactococcus lactis subsp. lactis S0 | CP010050.1 | From 2359456 to 2359947 |
| Lactococcus lactis subsp. lactis UC317 | LKLY01000004.1 | From 36130 to 36621 |
| Lactococcus lactis WG2 | LXWJ01000007.1 | From 37921 to 38412 |
| Lactococcus raffinolactis NBRC 100932 | BCVN01000102.1 | From 139 to 617 (forward) |
| Lactococcus piscium CNCM I-4031 | FLZT01000001.1 | From 149 to 628 (forward) |
| Lactococcus piscium MKFS47 | LN774769.1 | From 1708720 to 1709199 |
| Lactococcus garvieae 122061 | AP017373.1 | From 1356405 to 1356890 |
| Lactococcus garvieae 8831 | AFCD01000005.1 | From 510 to 995 (forward) |
| Lactococcus garvieae Lg-ilsanpaik- | JPUJ01000002.1 | From 180817 to 181302 |
| Lactococcus garvieae LG9 | AGQY01000137.1 | From 5631 to 6116 |
| Lactococcus garvieae M79 | FOTJ01000023.1 | From 3224 to 3709 |
| Lactococcus garvieae NBRC 100934 | BBJW01000010.1 | From 105946 to 106431 |
| Lactococcus garvieae PAQ102015-99 | LXWL01000009.1 | From 238437 to 238922 |
| Lactococcus garvieae TB25 | AGQX01000090.1 | From 28088 to 28573 |
| Lactococcus garvieae TRF1 | AVFE01000015.1 | From 42141 to 42626 |

As used herein a comX gene is understood to be a gene that encodes a functional ComX protein in the strain where it is expressed. By “functional ComX protein” it is meant a protein which induces or is able to induce the expression of genes regulated by the Com-box, and at least one of the late competence genes selected from comEA, comFA, comGA, dprA, coiA, ssbA, radA, radC, recA, and recX.

The ComX protein may have the amino acid sequence of SEQ ID NO:2, SEQ ID NO:4, SEQ ID NO: 6, SEQ ID NO:8, SEQ ID NO: 10, SEQ ID NO: 12, SEQ ID NO:14, SEQ ID NO:16, SEQ ID NO: 18, SEQ ID NO:20 or SEQ ID NO:22, or an amino acid sequence having at least 90%, at least 91%, at least 92%, at least 93%, at least 94%, at least 95%, at least 96%, at least 97%, at least 98%, or at least 99% identity to the amino acid sequence of SEQ ID NO:2, SEQ ID NO: 4, SEQ ID NO:6, SEQ ID NO:8, SEQ ID NO:10, SEQ ID NO:12, SEQ ID NO:14, SEQ ID NO: 16, SEQ ID NO:18, SEQ ID NO:20 or SEQ ID NO:22, or an amino acid sequence having at least 90%, at least 91%, at least 92%, at least 93%, at least 94%, at least 95%, at least 96%, at least 97%, at least 98%, or at least 99% similarity to the amino acid sequence of SEQ ID NO: 2, SEQ ID NO:4, SEQ ID NO:6, SEQ ID NO:8, SEQ ID NO:10, SEQ ID NO:12, SEQ ID NO: 14, SEQ ID NO: 16, SEQ ID NO: 18, SEQ ID NO:20 or SEQ ID NO:22.

In some embodiments, the ComX protein may have the amino acid sequence of SEQ ID NO:2, or an amino acid sequence having at least 90%, at least 91%, at least 92%, at least 93%, at least 94%, at least 95%, at least 96%, at least 97%, at least 98%, or at least 99% identity to the amino acid sequence of SEQ ID NO:2 or an amino acid sequence having at least 90%, at least 91%, at least 92%, at least 93%, at least 94%, at least 95%, at least 96%, at least 97%, at least 98%, or at least 99% similarity to the amino acid sequence of SEQ ID NO:2. In a particular embodiment, said ComX protein is used when the Lactococcus strain in step a) is a Lactococcus lactis subsp. lactis strain.

In some embodiments, the ComX protein may have the amino acid sequence of SEQ ID NO:4 or SEQ ID NO:6, or an amino acid sequence having at least 90%, at least 91%, at least 92%, at least 93%, at least 94%, at least 95%, at least 96%, at least 97%, at least 98%, or at least 99% identity to the amino acid sequence of SEQ ID NO:4 or SEQ ID NO:6 or an amino acid sequence having at least 90%, at least 91%, at least 92%, at least 93%, at least 94%, at least 95%, at least 96%, at least 97%, at least 98%, or at least 99% similarity to the amino acid sequence of SEQ ID NO:4 or SEQ ID NO:6. In a particular embodiment, said ComX protein is used when the Lactococcus strain in step a) is a Lactococcus lactis subsp. cremoris strain.

In some embodiments, the ComX protein may have the amino acid sequence of SEQ ID NO:8, or an amino acid sequence having at least 90%, at least 91%, at least 92%, at least 93%, at least 94%, at least 95%, at least 96%, at least 97%, at least 98%, or at least 99% identity to the amino acid sequence of SEQ ID NO:8 or an amino acid sequence having at least 90%, at least 91%, at least 92%, at least 93%, at least 94%, at least 95%, at least 96%, at least 97%, at least 98%, or at least 99% similarity to the amino acid sequence of SEQ ID NO:8. In a particular embodiment, said ComX protein is used when the Lactococcus strain in step a) is a

Lactococcus raffinolactis strain.

In some embodiments, the ComX protein may have the amino acid sequence of SEQ ID NO: 10, or an amino acid sequence having at least 90%, at least 91%, at least 92%, at least 93%, at least 94%, at least 95%, at least 96%, at least 97%, at least 98%, or at least 99% identity to the amino acid sequence of SEQ ID NO:10 or an amino acid sequence having at least 90%, at least 91%, at least 92%, at least 93%, at least 94%, at least 95%, at least 96%, at least 97%, at least 98%, or at least 99% similarity to the amino acid sequence of SEQ ID NO: 10. In a particular embodiment, said ComX protein is used when the Lactococcus strain in step a) is a Lactococcus plantarum strain.

In some embodiments, the ComX protein may have the amino acid sequence of SEQ ID NO: 12, or an amino acid sequence having at least 90%, at least 91%, at least 92%, at least 93%, at least 94%, at least 95%, at least 96%, at least 97%, at least 98%, or at least 99% identity to the amino acid sequence of SEQ ID NO:12 or an amino acid sequence having at least 90%, at least 91%, at least 92%, at least 93%, at least 94%, at least 95%, at least 96%, at least 97%, at least 98%, or at least 99% similarity to the amino acid sequence of SEQ ID NO: 12. In a particular embodiment, said ComX protein is used when the Lactococcus strain in step a) is a Lactococcus piscium strain.

In some embodiments, the ComX protein may have the amino acid sequence of SEQ ID NO: 14, SEQ ID NO:16 or SEQ ID NO:18, or an amino acid sequence having at least 90%, at least 91%, at least 92%, at least 93%, at least 94%, at least 95%, at least 96%, at least 97%, at least 98%, or at least 99% identity to the amino acid sequence of SEQ ID NO:14, SEQ ID NO: 16 or SEQ ID NO: 18 or an amino acid sequence having at least 90%, at least 91%, at least 92%, at least 93%, at least 94%, at least 95%, at least 96%, at least 97%, at least 98%, or at least 99% similarity to the amino acid sequence of SEQ ID NO:14, SEQ ID NO:16 or SEQ ID NO: 18. In a particular embodiment, said ComX protein is used when the Lactococcus strain in step a) is a Lactococcus garvieae strain.

In some embodiments, the ComX protein may have the amino acid sequence of SEQ ID NO: 20, or an amino acid sequence having at least 90%, at least 91%, at least 92%, at least 93%, at least 94%, at least 95%, at least 96%, at least 97%, at least 98%, or at least 99% identity to the amino acid sequence of SEQ ID NO:20 or an amino acid sequence having at least 90%, at least 91%, at least 92%, at least 93%, at least 94%, at least 95%, at least 96%, at least 97%, at least 98%, or at least 99% similarity to the amino acid sequence of SEQ ID NO: 20. In a particular embodiment, said ComX protein is used when the Lactococcus strain in step a) is a Lactococcus fujiensis strain.

In some embodiments, the ComX protein may have the amino acid sequence of SEQ ID NO: 22, or an amino acid sequence having at least 90%, at least 91%, at least 92%, at least 93%, at least 94%, at least 95%, at least 96%, at least 97%, at least 98%, or at least 99% identity to the amino acid sequence of SEQ ID NO:22 or an amino acid sequence having at least 90%, at least 91%, at least 92%, at least 93%, at least 94%, at least 95%, at least 96%, at least 97%, at least 98%, or at least 99% similarity to the amino acid sequence of SEQ ID NO: 22. In a particular embodiment, said ComX protein is used when the Lactococcus strain in step a) is a Lactococcus chungangensis strain.

According to the invention, when a ComX protein is defined by its amino acid sequence having a percentage of identity or percentage of similarity to a specific SEQ ID, said ComX protein is a functional ComX protein as defined herein.

By way of example and for the avoidance of doubt, in particular embodiments, where a ComX protein is specified as having a particular amino acid sequence, it is understood that the ComX protein comprises said amino acid sequence. In particular other embodiments, where a ComX protein is specified as having a particular amino acid sequence, it is understood that the ComX protein consists of said amino acid sequence.

In some embodiments, ComX proteins having percentage of identity or percentage of similarity as defined herein are selected from the list of protein sequences derived, after translation, from the list of DNA sequences disclosed in Table 1 above.

Preferably, reference to a sequence which has a percentage identity or similarity to any one of the SEQ ID NOs detailed herein refers to a sequence which has the stated percent identity or similarity with the SEQ ID NO referred to, over the entire length of the two sequences. Percentage (%) sequence identity is defined as the percentage of amino acids or nucleotides in a candidate sequence that are identical to the amino acids or nucleotides in a reference sequence, after aligning the sequences and introducing gaps, if necessary, to achieve the maximum percent sequence identity. Percentage (%) sequence similarity is defined as the percentage of amino acids in a candidate sequence that are similar to the amino acids in a reference sequence, after aligning the sequences and introducing gaps, if necessary, to achieve the maximum percent sequence similarity. Similarity between amino acids is based on established amino acid substitution matrices such as the PAM series (Point Accepted Mutation; e.g. PAM30, PAM70, and PAM250) or the BLOSUM series (BLOck Substitution Matrix; e.g. BLOSUM45, BLOSUM50, BLOSUM62, BLOSUM80, and BLOSUM90). Alignment for purposes of determining percent sequence identity or similarity can be achieved in various ways that are within the skill in the art, for instance, using publicly available computer software such as CLUSTALW, CLUSTALX, CLUSTAL Omega, BLAST, BLAST-2, ALIGN, ALIGN-2 or

Megalign (DNASTAR) software. In a particular embodiment, similarity between amino acids is determined using the BLASTp software with the BLOSUM62 matrix. Appropriate parameters for measuring alignment, including any algorithms needed to achieve maximal alignment over the full-length of the sequences being compared, or gap penalties to be introduced, can be determined by known methods.

In a particular embodiment, when the modulation in step b) results from an exogenous comX gene, said exogenous comX gene is (obtained) from a strain of the same species, in particular of the same subspecies, as the strain provided in step a). In any case, the exogenous comX gene needs to be functional, in particular needs to encode a functional ComX protein, as defined herein in the strain provided in step a).

### Exogenous DNA Polynucleotide

The method of the present invention comprises the step of contacting the strain of step b) with an exogenous DNA polynucleotide in a medium and incubating the resulting mixture for integration of the exogenous DNA into the genome of said strain [step c].

As used herein the term “exogenous DNA polynucleotide” refers to a DNA polynucleotide that is brought into the cytoplasm of said strain, in order to be integrated into the genome of said strain (target sequence).

In a particular embodiment, the method comprises carrying out step (b) [ComX modulation] and then carrying out step (c) [contact with the exogenous DNA polynucleotide] [i.e., that step (c) is carried out on a strain obtained following step (b)]. Thus, the method comprising the steps of:
(a) providing a strain of the Lactococcus genus, wherein said strain is transformable through natural competence;
(b) modulating the production of a ComX protein in said strain;
(c) contacting said strain obtained in step (b) with an exogenous DNA polynucleotide in a medium and incubating the resulting mixture for integration of the exogenous DNA polynucleotide into the genome of said strain; and
(d) selecting a strain which has integrated the exogenous DNA polynucleotide into its genome.

In another embodiment, the method comprises carrying out simultaneously step (b) [ComX modulation] and step (c) [contact with the exogenous DNA polynucleotide]. This option is appropriate when the ComX modulation is the result of the expression of the endogenous comX gene or of the increase of the expression of the endogenous comX gene of said strain. Thus, the method comprising the steps of:
(a) providing a strain of the Lactococcus genus, wherein said strain is transformable through natural competence;
(b) modulating the production of a ComX protein in said strain;
(c) contacting said strain with an exogenous DNA polynucleotide in a medium and incubating the resulting mixture for integration of the exogenous DNA polynucleotide into the genome of said strain; and
(d) selecting a strain which has integrated the exogenous DNA polynucleotide into its genome;
wherein step (b) and step (c) are carried out simultaneously.

In a particular embodiment, the sequence of the exogenous DNA polynucleotide used in step c) share some similarities or identities with the genome of the Lactococcus strain to be transformed (of step a). In a particular embodiment, the exogenous DNA polynucleotide used in step c) is designed such that its 5′ part and its 3′ part are identical or highly similar to parts of the genome of the Lactococcus strain to be transformed (of step a), while its central part can be different from the genome of the Lactococcus strain to be transformed (of step a). The high similarity of the arms with the regions surrounding the target sequence can be determined by the person skilled in the art using common general knowledge, in particular by reference to homologous recombination.

Thus, to replace a target sequence by a mutated sequence or a truncated sequence or a supplementary sequence in the genome of the Lactococcus strain to be transformed (of step a), the exogenous DNA polynucleotide used in step c) is designed such that:
its 5′ part is identical or highly similar to the region of the genome of the Lactococcus strain to be transformed which is on one side of the target sequence;
its central part contains the replacing sequence (i.e., the mutated sequence or the truncated sequence or the supplementary sequence); and its 3′ part is identical or highly similar to the region of the genome of the Lactococcus strain to be transformed which is on the other side of the target sequence.

The 5′ part and 3′ part are long enough to ensure efficient recombination. In a particular embodiment, each of the 5′ part and 3′ part is from 0.5 to 5 kb in length. The size of the arms can be determined by the person skilled in the art using common general knowledge, in particular by reference to homologous recombination.

In a particular embodiment, the exogenous DNA polynucleotide used in step c) is (obtained) from a strain of the Lactococcus genus.

In a particular embodiment, said exogenous DNA polynucleotide used in step (c) is (obtained) from a strain of the same species, in particular of the same subspecies, as the strain provided in step (a).

In a particular embodiment, the exogenous DNA polynucleotide used in step c) is from a strain of the Lactococcus lactis species. In a particular embodiment, the exogenous DNA polynucleotide used in step c) is from a strain of the same Lactococcus lactis subspecies as the strain provided in step a). In a particular embodiment, the exogenous DNA polynucleotide used in step c) is from a strain of a Lactococcus lactis subspecies which is different from the strain provided in step a).

In a particular embodiment, the exogenous DNA polynucleotide used in step c) is from a strain of the Lactococcus raffinolactis species

The exogenous DNA polynucleotide may encode part of a gene sequence, a gene sequence, or a plurality of gene sequences. The gene sequence may be operably linked to transcription regulator(s). In a particular embodiment, the exogenous DNA polynucleotide is linear. The exogenous DNA polynucleotide may be designed to facilitate its incorporation within the genome of the L. lactis strain by homologous recombination (e.g. the exogenous DNA polynucleotide may comprise one or more recombination arms). The exogenous DNA polynucleotide may be a single stranded linear DNA.

The exogenous DNA polynucleotide, when incorporated into the genome of said Lactococcus strain leads to genetic modification of the strain such as gene replacement (to add or to remove a mutation), gene addition (to add a new gene or to duplicate an existing gene), gene deletion (to remove part or the totality of a gene), modification of non-coding region (to modulate expression of a gene). Typically, the exogenous DNA polynucleotide, when incorporated into the genome of said Lactococcus strain confers an interesting or useful phenotype, e.g. modified kinetic of acidification, improved resistance to bacteriophage, modified capability to grow in milk, modified texturing properties, improved safety of the strain. For example, improved bacteriophage resistance could be achieved by incorporating genes coding for a restriction/modification system into the strain genome or by introducing a mutation or a deletion into the pip gene.

As an example, growth of a L. lactis strain in milk could be improved by inserting into the chromosome the prtP and prtM genes that allow casein hydrolysis and better nitrogen nutrition; alternatively, these genes could be inactivated to reduced milk proteolysis in cheese. hisDC and tyrDC are genes known to be responsible for biogenic amine production (histamine and tyramine, respectively) in a diversity of lactic acid bacteria; disruption or mutation of these genes could help to prevent safety issues related to cheese consumption.

In a particular embodiment, the exogenous DNA polynucleotide has a minimal size selected from the group consisting of 100 bp, 200 bp, 500 bp, 1 kb, 2 kb and 5 kb, and a maximal size selected from the group consisting of 500 bp, 1 kb, 2 kb, 5 kb, 10 kb, 20 kb and 50 kb. In a particular embodiment, the size of the exogenous DNA polynucleotide may be between 100 bp and 50 kb, more preferably between 500 bp to 20 kb, even more preferably between 1 kb to 10 kb.

The concentration of exogenous DNA polynucleotide in the medium of step (c) may be between 0.5 mg/L and 1 g/L, preferably between 1 mg/L and 500 mg/L, more preferably between 5 mg/L and 100 mg/L, even more preferably between 10 mg/L and 50 mg/L of medium.

### Selection of Transformed Strains

The method of the present invention comprises the step of selecting a strain which has integrated the exogenous DNA polynucleotide into its genome [step d)].

If needed, selection is carried out on some cells of colonies that have been previously obtained by multiplying, in the appropriate medium, cells obtained at the end of step c) (or at the end of the simultaneous steps b) and c), when appropriate).

Various methods for the selection of transformed bacteria are well known in the art (see, e.g. Sambrook et al.) and may be routinely applied by the person skilled in the art, such as PCR, DNA sequencing . . .

For example, when the exogenous DNA polynucleotide used in step c) provides a particular phenotype that the Lactococcus strain of step a) does not display (either a new phenotype or restoring a lost phenotype), it is possible to select strains which have integrated the exogenous DNA polynucleotide into their genome by selecting strains expressing the phenotype. This is the case for a strain having integrated in its genome an exogenous DNA polynucleotide mutated for the pip gene (that provides resistance to some bacteriophages).

For example, when the exogenous DNA polynucleotide used in step c) leads once integrated to a loss of a phenotype initially displayed by the Lactococcus strain of step a), it is possible to select strains which have integrated the exogenous DNA polynucleotide into their genome by selecting strains which do not diplay the phenotype any more. This is the case for an exogenous DNA polynucleotide bearing a mutated hisDC or tyrDC gene, which suppresses or decreases the production of histamine or tyramine, respectively.

As a particular example, the exogenous DNA polynucleotide may bear an antibiotic resistance gene. Accordingly, a Lactococcus strain which has integrated the exogenous DNA polynucleotide into its genome may be selected by plating onto a medium comprising said antibiotic. Only strains that express the appropriate antibiotic resistance gene, as a result of a successful transformation with the exogenous DNA polynucleotide, will multiply.

### Growth Conditions

As described in Example 3, a positive effect on natural competence induction in L. lactis strains was observed when cells were pre-cultured in a complex medium before transferring the cells to a chemically defined medium (FIG. 3).

Accordingly, in some embodiments the medium of step (c) is a chemically defined medium. As used herein, the term “chemically defined medium” (CDM) refers to a medium for which the exact chemical composition is known. Preferably, the CDM may have the composition of the CDM set out in Sissler et al. (1999, Proc Natl Acad Sci USA 96:8985-8990). Thus, in an embodiment, the chemically defined medium (CDM) comprises 0.5 g/L NH₄Cl, 9.0 g/L KH₂PO₄, 7.5 g/L K₂HPO₄, 0.2 g/L MgCl₂, 5 mg/L FeCl₂, 50 mg/L CaCl₂), 5 mg/L ZnSO₄, 2.5 mg/L CoCl₂, 0.05 g/L tyrosine, 0.1 g/L asparagine, 0.1 g/L cysteine, 0.1 g/L glutamine, 0.1 g/L isoleucine, 0.1 g/L leucine, 0.1 g/L methionine, 0.1 g/L tryptophan, 0.1 g/L valine, 0.1 g/L histidine, 0.2 g/L arginine, 0.2 g/L glycine, 0.2 g/L lysine, 0.2 g/L phenylalanine, 0.2 g/L threonine, 0.3 g/L alanine, 0.3 g/L proline, 0.3 g/L serine, 10 mg/L paraaminobenzoic acid, 10 mg/L biotin, 1 mg/L folic acid, 1 mg/L nicotinic acid, 1 mg/L panthotenic acid, 1 mg/L riboflavin, 1 mg/L thiamine, 2 mg/L pyridoxine, 1 mg/L cyanocobalamin, 5 mg/L orotic acid, 5 mg/L 2-deoxythymidine, 5 mg/L inosine, 2.5 mg/L dl-6,8-thioctic acid, 5 mg/L pyridoxamine, 10 mg/L adenine, 10 mg/L guanine, 10 mg/L uracil, 10 mg/L xanthine, and 5 g/L glucose . . .

In some embodiments, prior to step (c) said strain is incubated in a pre-culture medium, preferably wherein the pre-culture medium is a complex medium, more preferably wherein the pre-culture medium is M17G (i.e., the M17 medium supplemented with glucose) or THBG (i.e., the THB medium supplemented with glucose).

The complex medium may be Todd Hewitt broth (THB) (Todd and Hewitt, 1932; Updyke and Nickle, 1954) or M17 broth (Terzaghi and Sandine, 1975). THB may comprise 500 g/L beef heart infusion, 20 g/L peptic digest of animal tissue, 2 g/L dextrose, 2 g/L sodium chloride, 0.4 g/L sodium phosphate, 2.5 g/L sodium carbonate. M17 broth may comprise: 0.5 g/L ascorbic acid, 5 g/L lactose, 0.25 g/L magnesium sulfate, 5 g/L meat extract, 2.5 g/L meat peptone (peptic), 19 g/L sodium glycerophosphate, 5 g/L soya peptone (papainic), 2.5 g/L tryptone, 2.5 g/L yeast extract.

### Method for Identifying Strains Transformable by Natural Competence

In another aspect, the present invention relates to a method for identifying a strain of the Lactococcus genus which is transformable through natural competence. Said method comprises the following steps:
(a) providing a strain of the Lactococcus genus;
(b) transforming said strain with a plasmid expressing a comX gene having at least 90% identity, preferably having 100% identity, to the endogenous comX gene of said strain;
(c) contacting said strain obtained in step (b) with an exogenous marker DNA polynucleotide in a medium and incubating the resulting mixture for integration of the exogenous DNA polynucleotide into the genome of said strain; and
(d) determining the rate of recombination events;
   wherein a rate of at least 1×10⁻⁶ transformants per μg of DNA is indicative of a strain which is transformable through natural competence.

The term “rate of recombination events” may be used interchangeably with the term “transformation rate”. The rate of recombination events is calculated by determining the ratio of the number of cells having integrated the exogenous marker DNA polynucleotide over the total number of viable cells. A rate of at least 10⁻⁶ was selected as a threshold, based on the observation that the level of spontaneous mutation in lactococci is less than 10⁻⁶, typically around 10⁻⁷ mutants per μg of DNA [spontaneous means with no comX expression or overexpression].

By “at least 90% identity to the endogenous comX gene of said strain”, it is meant-as particular embodiments of the method-at least 91%, at least 92%, at least 93%, at least 94%, at least 95%, at least 96%, at least 97%, at least 98%, or at least 99% identity. In a particular embodiment, said comX gene has 100% identity to the endogenous comX gene of said strain.

In a particular embodiment, said method is implemented with a strain of the Lactococcus genus selected from the group consisting of Lactococcus lactis, Lactococcus raffinolactis, Lactococcus plantarum, Lactococcus piscium, Lactococcus garivieae, Lactococcus fujiensis and Lactococcus chungangensis.

In a particular embodiment, said method is implemented with a strain of the Lactococcus lactis species. Said method comprises the following steps:
(a) providing a strain of the Lactococcus lactis species;
(b) transforming said strain with a plasmid expressing a comX gene having at least 90% identity to the polynucleotide sequence of SEQ ID NO:1, 3 or 5;
(c) contacting said strain obtained in step (b) with an exogenous marker DNA polynucleotide in a medium and incubating the resulting mixture for integration of the exogenous DNA polynucleotide into the genome of said strain; and
(d) determining the rate of recombination events;
   wherein a rate of at least 1×10⁻⁶ transformants per μg of DNA is indicative of a strain of the Lactococcus lactis species which is transformable through natural competence.

In a particular embodiment, said method is implemented with a strain of the Lactococcus raffinolactis species. Said method comprises the following steps:
(a) providing a strain of the Lactococcus raffinolactis species;
(b) transforming said strain with a plasmid expressing a comX gene having at least 90% identity to the polynucleotide sequence of SEQ ID NO:7;
(c) contacting said strain obtained in step (b) with an exogenous marker DNA polynucleotide in a medium and incubating the resulting mixture for integration of the exogenous DNA polynucleotide into the genome of said strain; and
(d) determining the rate of recombination events;
   wherein a rate of at least 1×10⁻⁶ transformants per μg of DNA is indicative of a strain of the Lactococcus lactis species which is transformable through natural competence.

In some embodiments, the comX gene is from a strain of the same species, in particular of the same subspecies, as the strain provided in step a). In some embodiments, the comX gene is identical (100% identity) to the polynucleotide sequence of the endogenous comX gene of the strain of step a).

In some embodiments, when the strain of step a) is a Lactococcus lactis subsp. lactis strain, the comX gene has at least 90%, at least 91%, at least 92%, at least 93%, at least 94%, at least 95%, at least 96%, at least 97%, at least 98%, at least 99%, or 100% identity to the polynucleotide sequence of SEQ ID NO:1.

In some embodiments, when the strain of step a) is a Lactococcus lactis subsp. cremoris strain the comX gene has at least 90%, at least 91%, at least 92%, at least 93%, at least 94%, at least 95%, at least 96%, at least 97%, at least 98%, at least 99%, or 100% identity to the polynucleotide sequence of SEQ ID NO:3 or SEQ ID NO:5.

In some embodiments, when the strain of step a) is a Lactococcus raffinolactis strain the comX gene has at least 90%, at least 91%, at least 92%, at least 93%, at least 94%, at least 95%, at least 96%, at least 97%, at least 98%, at least 99%, or 100% identity to the polynucleotide sequence of SEQ ID NO:7.

In some embodiments, when the strain of step a) is a Lactococcus plantarum strain the comX gene has at least 90%, at least 91%, at least 92%, at least 93%, at least 94%, at least 95%, at least 96%, at least 97%, at least 98%, at least 99%, or 100% identity to the polynucleotide sequence of SEQ ID NO:9.

In some embodiments, when the strain of step a) is a Lactococcus piscium strain the comX gene has at least 90%, at least 91%, at least 92%, at least 93%, at least 94%, at least 95%, at least 96%, at least 97%, at least 98%, at least 99%, or 100% identity to the polynucleotide sequence of SEQ ID NO:11.

In some embodiments, when the strain of step a) is a Lactococcus garvieae strain the comX gene has at least 90%, at least 91%, at least 92%, at least 93%, at least 94%, at least 95%, at least 96%, at least 97%, at least 98%, at least 99%, or 100% identity to the polynucleotide sequence of SEQ ID NO:13, SEQ ID NO:15, or SEQ ID NO:17.

In some embodiments, when the strain of step a) is a Lactococcus fujiensis strain the comX gene has at least 90%, at least 91%, at least 92%, at least 93%, at least 94%, at least 95%, at least 96%, at least 97%, at least 98%, at least 99%, or 100% identity to the polynucleotide sequence of SEQ ID NO:19.

In some embodiments, when the strain of step a) is a Lactococcus chungangensis strain the comX gene has at least 90%, at least 91%, at least 92%, at least 93%, at least 94%, at least 95%, at least 96%, at least 97%, at least 98%, at least 99%, or 100% identity to the polynucleotide sequence of SEQ ID NO:21.

It is preferable to use, as an exogenous marker DNA polynucleotide, a polynucleotide bearing a gene which is initially not present in the Lactococcus strain of step a) [even as a mutated version]. This would avoid that during step c) the Lactococcus strain acquires a functional gene by other means than natural competence, e.g. by spontaneous mutation of its genome.

As an example, the exogenous marker DNA polynucleotide bears a gene encoding a luciferase gene. Accordingly, a Lactococcus strain which has integrated the exogenous DNA polynucleotide into its genome may be selected for expression of the luciferase. Only strains that express the luciferase gene (i.e., integrated) will be detectable by bioluminescence.

As another example, the exogenous marker DNA polynucleotide bears an antibiotic resistance gene. Accordingly, a Lactococcus strain which has integrated the exogenous DNA polynucleotide into its genome may be selected by plating the cells onto a medium comprising said antibiotic.

An example of a method for identifying a strain of the Lactococcus genus which is transformable through natural competence according to the present invention (Assay A) may be performed using the following steps:
i) Providing a strain of the Lactococcus genus, in particular of the Lactococcus lactis species.
ii) Transforming said strain with a plasmid expressing a comX gene having at least 90% identity, preferably having 100% identity to the endogenous comX gene of said strain (e.g. the pGhP32comX_{MG} plasmid of Materials and Methods).
iii) Pre-culturing the transformed strain overnight in a complex medium supplemented with glucose (e.g. M17G) at 30° C.
iv) Diluting about 1.5 mL of the pre-culture in about 8.5 mL of fresh medium.
v) After about 2 hours further growth at 30° C., washing the cells twice with distilled water and adjusting the OD₆₀₀ to 0.05 in a chemically defined medium (e.g. CDM) containing 5 μg mL⁻¹ erythromycin and an osmo-stabilizer (e.g. 5% [v/v] glycerol or 5% [w/v] mannitol).
vi) Adding 5 μg of exogenous DNA polynucleotide bearing an antibiotic resistance gene to 300 μl of the culture medium (e.g. the 3.7 kb PCR product generated from the pGEMrpsL plasmid as described in Materials and Methods).
vii) Incubating the resulting culture for about 6 hours at 30° C.
viii) Plating the cells onto agar plates comprising the complex medium supplemented with glucose (e.g. M17G) and appropriate antibiotic (i.e. corresponding to the antibiotic resistance gene of the exogenous DNA polynucleotide) and incubating for about 48 hours.
ix) Counting the colony forming units (CFU) and determining the transformation rate, wherein a transformation rate of at least 1×10⁻⁶ transformants per μg of DNA is indicative of a strain which is transformable through natural competence.

The transformation rate may be calculated as the number of antibiotic-resistance CFU mL⁻¹ divided by the total number of viable CFU mL⁻¹.

Various preferred features and embodiments of the present invention will now be described by way of non-limiting examples.

### Examples

### Example 1: Induction of the comGA Promoter by Constitutive comX Expression in Various Strains of the Lactococcus Species

a) in Lactococcus lactis subsp. cremoris strains (MG1363 and KW2)

To test the ability of ComX to induce the late competence genes in Lactococcus lactis subsp. cremoris strains, a constitutive comX expression plasmid (pGhP32comX_{MG}) was created by cloning the comX gene from strain MG1363, under the control of the lactococcal P₃₂ promoter on the thermosensitive plasmid pG⁺host9. The latter was introduced in strain KW2 that contains a chromosomally-encoded P_{comGA[MG]}-luxAB transcriptional fusion (BLD101). The promoter of the late competence gene comGA (P_{comGA}) contains a putative ComX-binding motif and is used here as proxy for competence activation in the ComX⁺strain.

As alternative for more resistant strains to electro-transformation, and subsequently to chromosome integration, a portable luminescent reporter system was also constructed. This replicative plasmid carries the luminescent reporter P_{comGA[MG]}-luc with the P₃₂-COMX_{MG} cassette. The pGhP32comX_{MG}-P_{comGA[MG]}-luc plasmid was transformed in strain MG1363.

Specific P_{comGA[MG]}-luc/luxAB activities were monitored for the different strains constructed. The luminescent assays were performed in rich (M17G) and/or CDM media comparing the luciferase activity between the overexpressing comX strain and its related negative control (no additional comX copy).

In the KW2 strain containing the P_{comGA[MG]}-luxAB reporter as mono-copy in their chromosome, specific luciferase activity was observed for KW2 containing the P₃₂-comX cassette allowing the constitutive production of ComX. This confirms that comX expression can be carried out in various L. lactis subsp. cremoris strains using an exogenous comX gene obtained from the same strain or from a strain of the same subspecies. Eight recombinant clones of the KW2 ComX+reporter strain were randomly selected and their specific luciferase activity was monitored in CDM growth conditions. This medium was chosen because it was shown to be permissive for competence development in various streptococcal species. To ensure reproducibility of the assay, exponentially-growing cells in complex medium (M17 conditions) were washed and inoculated in fresh CDM before starting the experiment. As expected, all tested ComX+clones (cl01 to cl08) displayed between 101- and 10⁴-fold higher specific luciferase (Lux) activity than the control strain carrying the empty vector (FIGS. 2A and 2B).

Similar results were obtained with the portable luminescent reporter systems in MG1363 (FIG. 2C).

These results strongly suggest that, in the L. lactis subsp. cremoris strains MG1363 and KW2, ComX induces the comG operon. Additionally, these observations validate these reporter fusions (both chromosomal and plasmid-borne) as a tool to identify conditions capable to activate the comG-operon which is essential to natural transformation.

b) in a L. lactis subsp. lactis strain (IL1403)

A constitutive comX expression plasmid (pGhP32comX_{IO}) was created by cloning the comX gene from strain IO-1, under the control of the lactococcal P₃₂ promoter on the thermosensitive plasmid pG⁺host9. A portable luminescent reporter system was also constructed; this replicative plasmid carries the luminescent reporter P_{comGA[IO]}-luc with the P₃₂-comX_{IO} cassette. The promoter of the late competence gene comGA (P_{comGA}) contains a putative ComX-binding motif and is used here as proxy for competence activation in the ComX+strain.

This replicative plasmid pGhP32comX_{IO}-P_{comGA[IO]}-luc was transformed in strain IL1403 and specific P_{comGA[IO]}-luc activities were monitored. One of the IL1403 transformants produced specific P_{comGA[IO]}-luc activities confirming that ComX induces the comG operon (FIG. 2D).

### Example 2: Analysis of Essential Late Com Genes Present in L. lactis Genomes

Among L. lactis strains, genomic variability was previously investigated for comX and dprA alleles (Wydau et al., 2006). While all strains (31/31) display a complete version of comX, the dprA content is variable among subspecies: 50% of the lactis strains (10/20) contain nonsense mutations in dprA while all cremoris strains (11/11) harbor an intact and potentially functional dprA gene.

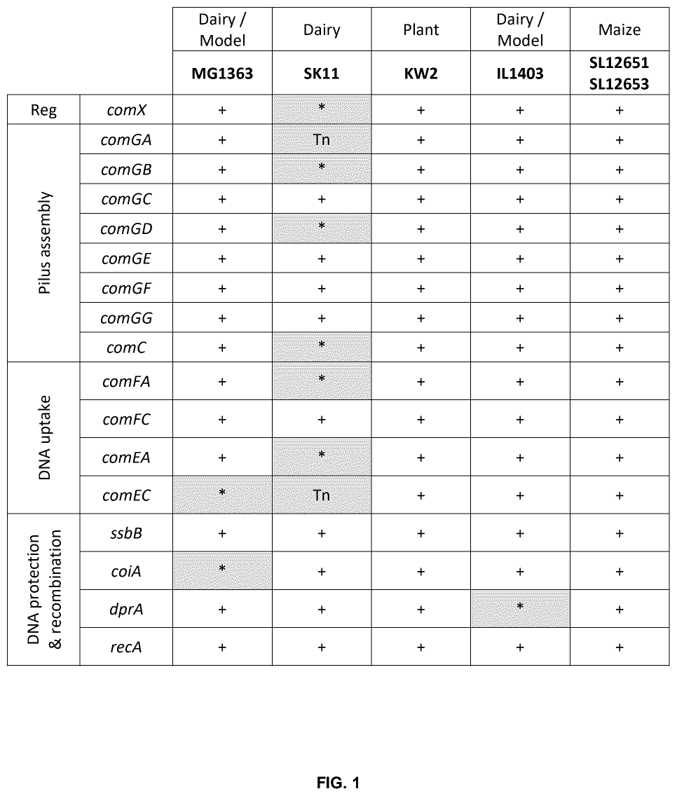

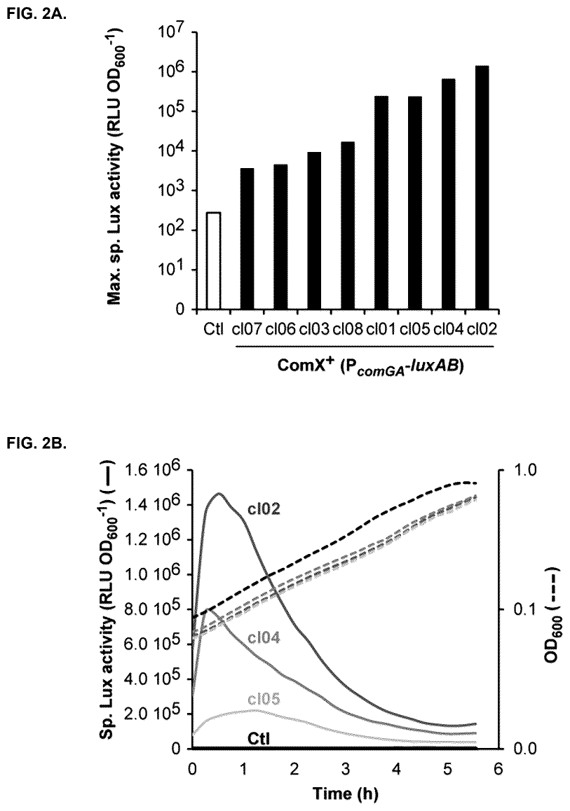

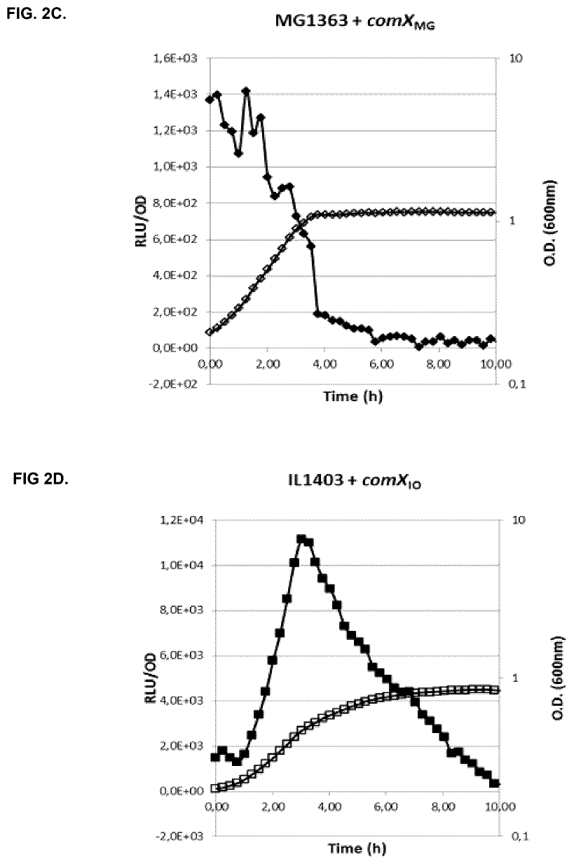

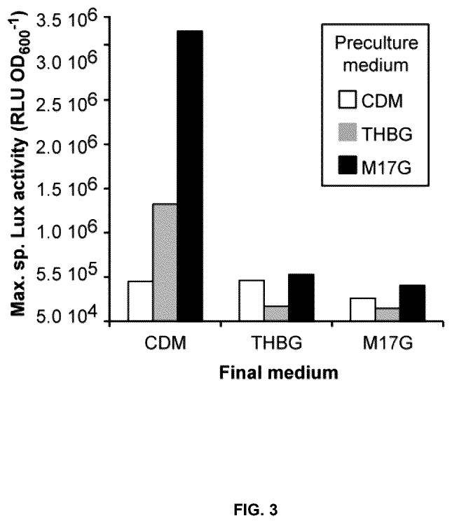

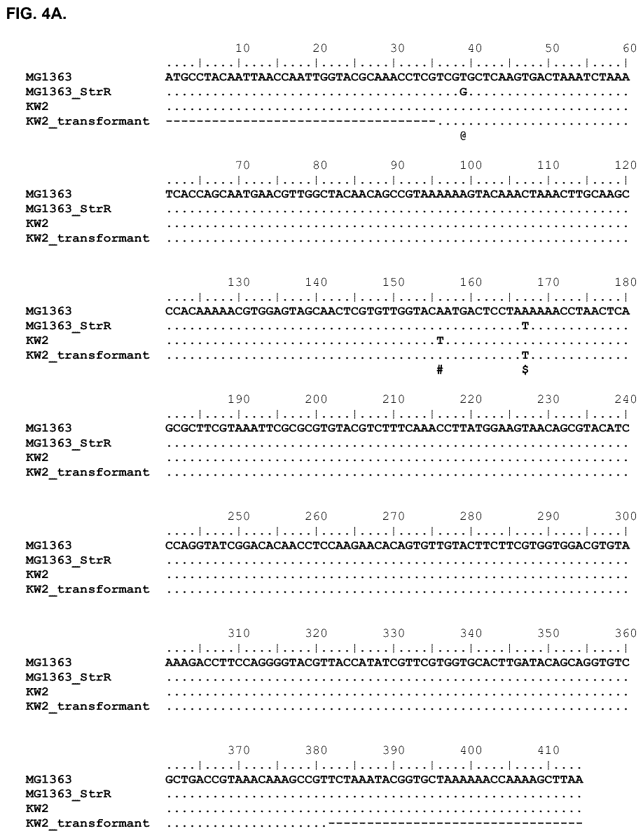

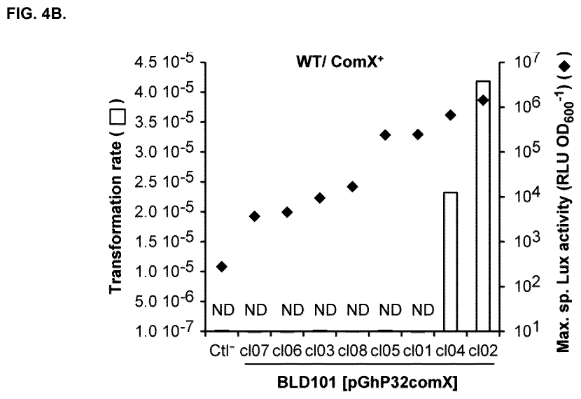

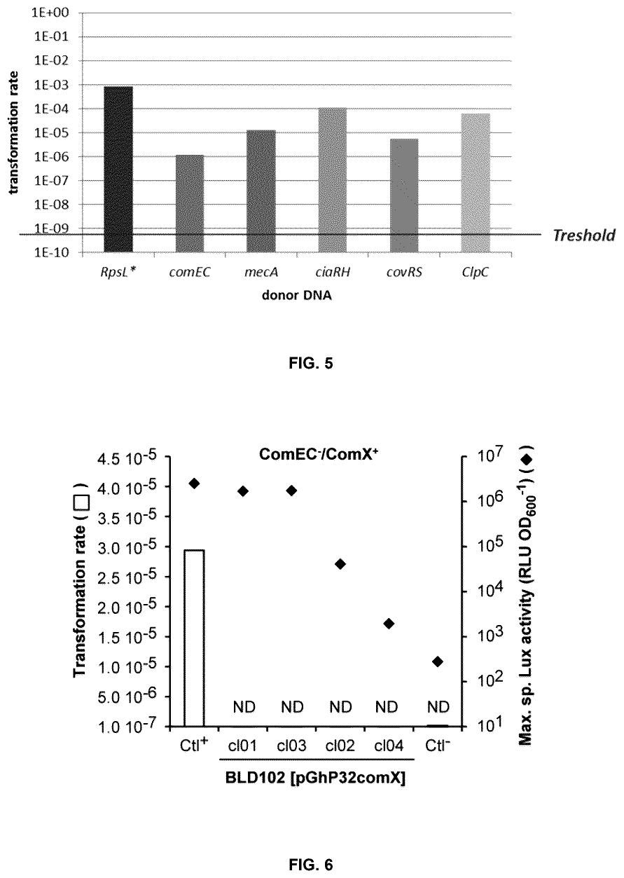

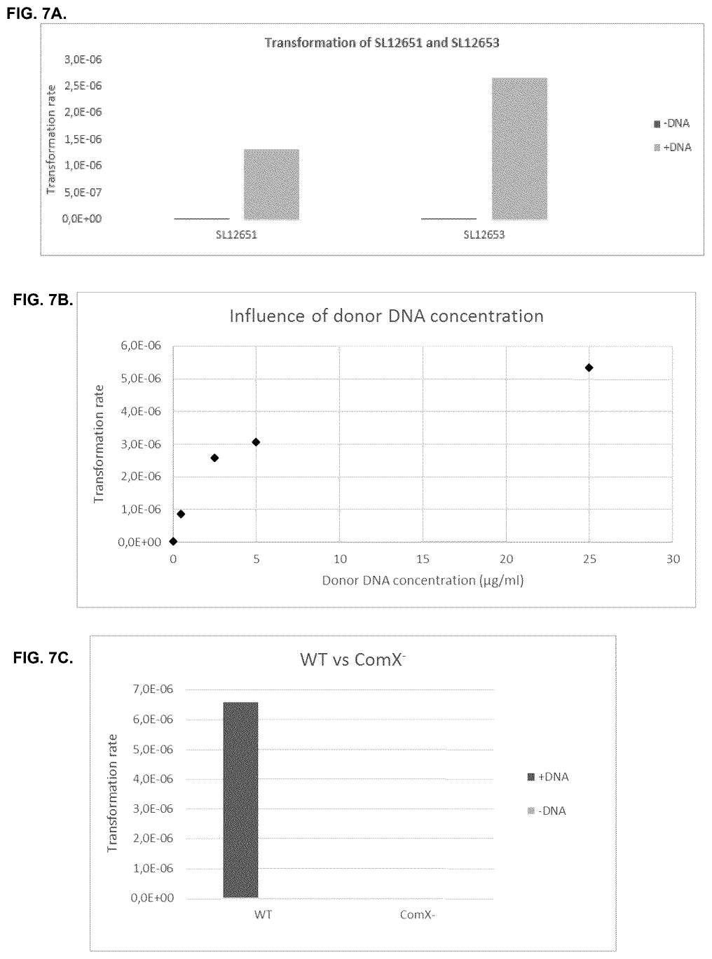

Since dprA is hypothesized to be important in the natural competence mechanism, its integrity in L. lactis strains prompted us to further analyze the minimal set of late com genes (17 candidate genes including comX; FIG. 1) in the genomes of 3 subsp. cremoris strains and 1 subsp. lactis strain which are publicly available (strains MG1363, SK11, KW2 and IL1403). This in silico analysis reveals that the genome of SK11 contains a high number of pseudogenes in key competence genes (between 5 and 8 incomplete late com genes) due to transposon insertion or frameshifting events (nucleotide(s) insertion or deletion). In particular, the presence of transposable elements in comGA and/or comEC genes, which are respectively essential for pilus assembly and DNA transport, strongly suggests that natural transformation is no more functional in those strains. Although the set of full-length competence genes in the laboratory strain MG1363 is larger, mutations in comEC (nucleotide insertion) and coiA (nonsense mutation) probably impair its ability to transform DNA by competence (Wegmann et al., 2007). Those mutations were also found in the genome of its isogenic derivative NZ9000, which strongly suggests that they do not result from DNA sequencing errors. As far as the L. lactis subsp. lactis IL1403 strain is concerned, its dprA gene contains nonsense mutations probably impairing its ability to transform DNA by competence. In contrast, strain KW2 of plant origin (corn fermentation) contains the whole set of known essential late genes required to fulfil natural DNA transformation, making it the best candidate to further study the functionality of competence in the cremoris subspecies. Two other strains from our collection, L. lactis subsp. lactis SL12651 and SL12653, were also found to contain the whole set of known essential late genes (FIG. 1).

### Example 3: Effect of Growth Conditions on ComX Activation

We investigated the effect of pre-culturing and culturing conditions (M17G, THBG, and CDM) on the activation of the reporter fusion in the ComX⁺strain. For this purpose, clone 02 (FIG. 2A) was selected since it exhibits the strongest Lux activity. Interestingly, more than a 20-fold variation in the maximum Lux activity was dependent on the pre- and culturing medium which was used (FIG. 3). Particularly, a positive impact of the transition of pre-culture cells from a complex medium to a defined medium was observed. The highest specific Lux activity (˜3×10⁶ RLU OD₆₀₀⁻¹) was obtained for a switch from M17G to CDM, followed by THBG to CDM, while all other combinations gave lower activities (between ˜ 1.5× and 5.5×10⁵ RLU OD₆₀₀⁻¹). This indicates that first a chemically defined medium is superior for maximizing activation of late com genes of L. lactis KW2 than complex rich media, but also that the switch from complex medium (e.g. M17G or THBG) to defined medium is critical.

Together, these results show that ComX is functional in strain KW2 when it is constitutively produced (i.e. expressed) and that growth conditions have a significant impact on the activation of late com genes.

### Example 4: Constitutive comX Expression Induces Natural Transformation

a) Acquisition of Single Mutations in the KW2 Genome from Exogenous DNA

We first tested the transfer of single point mutations in the chromosome of the ComX+KW2 strain. The transforming PCR fragments used encompass the mutated rpsL allele of a spontaneous streptomycin-resistant (Str′) clone of L. lactis subsp. cremoris MG1363 (strA1 allele, also called rpsL*). This mutated allele bears an A→T substitution at position 167 [resulting in the altered ribosomal protein S12 with mutation K56I] as compared to the sequence of the wild-type, streptomycin-sensitive MG1363. In addition to this mutation, the two rpsL alleles differ by a silent nucleotide substitution at position 39 (T→G). The sequence of the rpsL (wild-type) and rpsL* (conferring streptomycin resistance) alleles are disclosed respectively as SEQ ID NO:23 and NO: 24 (FIG. 4A). Independently of these two substitutions located at positions 39 and 167, the rpsL alleles of KW2 and MG1363 differ by a nucleotide substitution at position 156 (A in MG1363, T in KW2). The rpsL allele of KW2 is disclosed as SEQ ID NO:25 (FIG. 4A). To ensure efficient recombination, the transforming PCR product also contains upstream and downstream recombination arms of ˜1.85 kb surrounding the strA1 mutation. Transformation assays were performed with the eight previously selected clones of the ComX⁺reporter strain (BLD101 [pGhP32comX_{MG}]) and the control strain (BLD101 [pG⁺host9], empty vector) using the standard protocol reported in Material and Methods. Validation of natural transformation is made by sequencing the rpsL region covering the point mutations from the donor DNA conferring streptomycin resistance using primers RpsL Univ UP and RpsL Univ DN.

Remarkably, the ComX⁺clones 02 and 04 that displayed the highest P_{comGA} activation (≥7×10⁵ RLU OD₆₀₀⁻¹) yielded mutation frequencies ˜15-fold higher than the background level of spontaneous mutation that was calculated in the absence of DNA (FIG. 4B). After subtraction of the background, a transformation rate of up to 4×10⁻⁵ transformants per μg of DNA (˜10⁴ transformants ml⁻¹) was obtained for clone 02 which displays the highest P_{comGA} activation. In contrast, the negative control strain had a spontaneous mutation rate of ˜1×10⁷ transformants per μg of DNA.

The rpsL ORF of 10 Str′-derivatives of cl02 was amplified by PCR and sequenced. In all cases, we observed the co-transfer of strA1 (mutation A→T at position 167 of the rpsL gene) and the closely-located T→A mutation at position 156. In some cases, the T→G mutation at position 39 was also co-transferred with strA1. The chimeric nature of rpsL in some Str^{r} ComX+derivatives of KW2 (i.e. presence of both mutations at positions 156 and 167 without the mutation at position 39) ultimately demonstrates that a recombination process occurred between the exogenous and chromosomal DNA (FIG. 4A). In contrast, this rearrangement was not observed in the rpsL gene of spontaneous Str′ mutants obtained in the negative control experiments (i.e. assays performed in absence of exogenous DNA, or with the control strain carrying the empty vector in presence of exogenous DNA). These results show that exogenous DNA can enter KW2 cells and be integrated in their chromosome by homologous recombination when a certain threshold of comX expression is reached.

b) Construction of Deletion Mutants by Natural Competence in L. lactis Subsp. Cremoris KW2 Overexpressing comX

The previous result (Example 4, section a) strongly suggests that DNA transfer occurs in L. lactis KW2. The 3 mutations transferred by natural transformation are grouped on a 128-bp fragment. If a longer DNA fragment could be similarly integrated in the L. lactis chromosome remains to be determined.

We wondered if overlap PCR as donor DNA could equivalently allow gene insertions or gene deletions. The idea was to replace the target gene by an antibiotic resistance cassette, i.e. the chloramphenicol resistance cassette P₃₂-cat. For this purpose, a DNA fragment was constructed by overlap PCR containing the P₃₂-cat cassette flanked by two homologous arms (minimum ˜ 1.5 kb) containing the upstream and downstream regions of the targeted gene.

To this end, exogenous DNA polynucleotides containing P₃₂-cat surrounded by KW2-specific recombination arms (˜ 1.5 kb) were assembled in vitro by overlapping PCR to target the comEC, mecA, ciaRH, covRS or clpC gene (see Materials and Methods for details) and transferred by natural transformation in the ComX+strain (cl02). Validation of natural transformation is made by sequencing the targeted region (comEC, mecA, ciaRH, covRS or clpC, which should contain the chloramphenicol resistance cassette P₃₂-cat) using primers listed in Table 3.

The transformation rate observed for overlap PCR products was ˜ 1.2×10⁻⁶ to 1.1×10⁻⁴ transformants per μg of DNA for the different overlap DNA fragments that were tested (see FIG. 5). Compared to the transformation rate observed for the exchange of a homologous DNA fragment containing only three point mutations (rpsL* donor DNA; 8×10⁻⁴ transformants per μg of DNA), these rates are relatively high for DNA double recombination deletion/replacement.

### Example 5: a KW2 ΔcomEC Mutant is Unable of Natural Competence Transformation

To confirm that the observed horizontal DNA transfer in ComX+KW2 cells was indeed mediated by natural competence, and not by phage transduction or conjugation, we investigated the role of the ComEC protein, which is essential for the uptake of transforming DNA through the cell membrane (the comEA gene, together with the comFA, comGA, dprA, coiA, ssbA, radA, radC, recA and recX genes are preceded by a Com-box and have been found to be activated in KW2 following constitutive comX expression; data not shown).

To create the ΔcomEC strain, clone 02 of the ComX+reporter strain, which was tested above, was grown in CDM conditions in presence of PCR products encompassing the comEC gene disrupted by the insertion of the chloramphenicol resistance cassette P₃₂-cat (see Materials and Methods). Four mutants with disrupted comEC (BLD102 [pGhP32comX_{MG}] cl01 to cl04) were validated by PCR for P₃₂-cat insertion in comEC. Transformation assays with the mutated rpsL allele showed that the frequencies of appearance for Str′ clones in all tested ΔcomEC derivatives were similar to the background level of spontaneous rpsL mutation frequencies (<10⁻⁷) (FIG. 6). Although heterogeneity in P_{comGA} activation was observed between clones as previously reported for the WT ComX+reporter strain, half of the ΔcomEC derivative clones (i.e. cl01 and cl03) displayed maximum specific Lux activity similar to the transformable WT strains (>1.0×10⁶ RLU OD₆₀₀⁻¹) (FIG. 4B). This shows that the transformation defect in these ΔcomEC clones does not result from a too low production of ComX.

Taken together, these results demonstrate that natural DNA transformation could be activated by ComX overexpression in L. lactis subsp. lactis KW2. Moreover, to the best of our knowledge, these data provide the first ever experimental evidence of transformation of L. lactis by natural competence.

### Example 6: Natural Competence in Two Strains of the L. raffinolactis Species

Following the positive results obtained regarding natural competence in Lactococcus lactis strains, other strains of the Lactococcus genus were tested. Two strains of L. raffinolactis were able to capture plasmid pGhost-Core (15 μg/300 μl) used as donor DNA: LMG13098 and LMG14164. These results suggest that these two strains of L. raffinolactis are naturally competent for plasmid transformation and that, in these strains, natural competence is independent of artificial comX-overexpression.

The fact that another Lactococcus species could be transformed by competence opens additional possibilities for industrial applications.

### Example 7: Transformation by Natural Competence in 2 Lactococcus lactis Subsp. Lactis Strains

Two Lactococcus lactis subsp. lactis strains, SL12651 and SL12653, carrying all the essential late com genes (FIG. 1) were tested. As donor DNA, PCR fragments which encompass the mutated rpsL allele (rpsL*) of a spontaneous streptomycin-resistant (Str′) clone of L. lactis subsp. lactis IL1403 was used. Cells were pre-cultured overnight in a complex medium supplemented with glucose (e.g. M17G) at 30° C. Cells were washed twice with distilled water and inoculated at an OD600 of 0.05 in 200 μl M17G containing 25 μg mL⁻¹ donor DNA rpsL*. After 24 hours of culture at 30° C., cells incubated or not with donor DNA were spread onto agar plates comprising the complex medium supplemented with glucose (e.g. M17G) and appropriate antibiotic (i.e. streptomycin). CFUs were counted after 48 hours of incubation at 30° C. Remarkably, SL12651 and SL12653 yielded a transformation rate of up to 1×10⁻⁶ of DNA when grown in M17G rich medium (FIG. 7A; +DNA). In contrast, the negative control in absence of donor DNA had a spontaneous mutation rate of 6×10⁻⁹ (FIG. 7A;-DNA). The transformants were validated by sequencing the rpsL region covering the point mutation from the donor DNA conferring streptomycin resistance.

Then, the SL12653 strain was assayed in the same conditions with variable quantity of donor DNA (0.5, 2.5, 5 and 25 μg mL⁻¹). It has been shown that the transformation rate obtained is directly correlated to the initial quantity of donor DNA, yielding up to a transformation rate of 5×10⁻⁶ (FIG. 7B).

Moreover, to confirm that the observed horizontal DNA transfer was mediated by natural competence, the comX gene of SL12653 was knocked-out (as described in example 5 above). Three mutants of SL12653 with disrupted comX gene were designed by inserting PCR products encompassing the comX gene disrupted by the insertion of the chloramphenicol resistance cassette P₃₂-cat and validated by PCR for P₃₂-cat insertion. Transformation assays with rpsL* as donor DNA in all AcomX clones (ComX) showed that the frequencies of appearance of Str′ clones were similar to the background level of spontaneous mutation frequencies (FIG. 7C). These results confirm that in SL12653, the transformation is dependent on the expression of the endogenous comX gene.

Finally, the transformability of the SL12653 strain was also assayed by overexpressing the comX gene. Thus, an inducible comX expression plasmid [pGhPxylTcomX_{IO}] was constructed by cloning the comX gene from strain L. lactis subsp. lactis IO-1 under the control of the P_{xyIT} promoter from strain IO-1 on the thermosensitive plasmid pG⁺host9. This plasmid is a variant of pGhPxylTcomX_{MG} (pGIFPT001) described in David et al., 2017. The transformation procedure described in David et al (2017) was followed. In presence of xylose (1%), SL12653 [pGhPxylTcomX_{IO}] yielded a transformation rate at least 20-fold higher than in absence of xylose, confirming that the overexpression of comX in SL12653 increased its transformability by natural competence.

### Materials and Methods

### Bacterial Strains, Plasmids, and Growth Conditions

The bacterial strains and plasmids used in this application are listed in Table 2.

TABLE 2
list of used bacterial strains and plasmids

| | | |
|---|---|---|
| Strain or plasmid | Characteristics ^{a} | Source or reference |

E. coli

| | | |
|---|---|---|
| TG1 | supE hsdΔ5 thi Δ(lac-proAB) | Sambrook, J., E. F. Fritsch, and T. |
| | F′[traD36 proAB⁺ lacI^{q} lacZΔM15] | Maniatis. 1989. Cold Spring Harbor |
| | | Laboratory, Cold Spring Harbor, N. Y. |
| EC1000 | Km^{r}, recΔ⁺; MC1000 containing a | G. Venema, and K. Leenhouts. 1995. J. |
| | copy of the repA gene from pWV01 | Law, J., G. Buist, A. Haandrikman, J. Kok, |
| | in its chromosome | Bacteriol. 177: 7011-7018. |

L. lactis

| | | |
|---|---|---|
| MG1363 | Laboratory strain, dairy origin | Gasson, M. J. 1983. J. Bacteriol. 154: 1-9. |
| KW2 | Wild-type isolate from corn | Kelly, W. J., E. Altermann, S. C. Lambie, |
| | fermentation | and S. C. Leahy. 2013. Front Microbiol. |
| | | 4: 257. |
| IL1403 | Laboratory strain, dairy origin | Chopin, A., M. C. Chopin, A. Moillo-Batt, |
| | | and P. Langella. 1984. Plasmid 11: 260- |
| | | 263 |
| IO-1 | Wild-type isolate from water in the | Ishizaki A, Osajima K, Nakamura K, |
| | drain pit of a kitchen sink | Katsunori K, Hara T, and Ezaki T. 1990. |
| | | J. Gen. Appl. Microbiol., 36, 1-6 |
| SL12651 | Wild-type isolate from plant | DuPont/Danisco collection |
| SL12653 | material (maize) | |
| BLD101 | KW2 kw2_0563::P_{comGA[MG]}-luxAB | This application |
| BLD102 | BLD101 comEC::P₃₂-cat | This application |
| BLD107 | BLD101 mecA::P₃₂-cat | This application |
| BLD108 | BLD101 ciaRH:P₃₂-cat | This application |
| BLD109 | BLD101 covRS:P₃₂-cat | This application |
| BLD105 | BLD101 clpC:P₃₂-cat | This application |

L. raffinolactis

| | | |
|---|---|---|
| LMG13098 | Wild-type isolate from garden | LMG collection |
| | carrots | |
| LMG14164 | Wild-type isolate from goose | LMG collection |

Plasmids

| | | |
|---|---|---|
| pGEM ®-T easy | Ap^{r}; cloning vector | Promega |
| pG⁺host9 | Em^{r} Ts | Maguin, E., H. Prevost, S. D. Ehrlich, and |
| | | A. Gruss. 1996. J. Bacteriol. 178: 931-935 |
| pGhost-Core | Em^{r} Ts; pG⁺host9 derivative | This application |
| | containing the Core part of the | |
| | resolution site IRS recognized by | |
| | the Tnpl from Tn4430 | |
| pMG36eT | Em^{r}; E. coli-L. lactis shuttle vector | Fontaine, L. and P. Hols. 2008.Appl. |
| | containing the P₃₂ constitutive | Environ. Microbiol. 74: 1102-1110. |
| | promoter from L. lactis | |
| pJIM4900 | Em^{r} Ts; pG⁺host9 derivative | E. Guédon, (laboratory collection) |
| | containing the luxAB genes of | |
| | Photorhabdus luminescens | |
| pXL | Em^{r}; pTRKH2 derivative containing | Blomqvist T, Steinmoen H, Håvarstein |
| | the luc reporter gene | LS. |
| | | Appl Environ Microbiol. 2006. |
| | | October; 72(10): 6751-6. |
| PSEUDOPusp45GFP | Em^{r}; suicide vector containing the | usp45-Kovacs, J. Kok, O. P. Kuipers, and J. W. |
| | Ilmg_pseudo_10(kw2_0563)::Pᵤₛₚ₄₅- | Overkamp, W., K. Beilharz, W. R. Detert |
| | gfp⁺ insertion cassette | Oude, A. Solopova, H. Karsens, A. |
| | | Veening. 2013. Appl. Environ. Microbiol. |
| | | 79: 6481-6490. |
| pUC18Cm | Ap^{r} Cm^{r}; pUC18 derivative | Goffin, P., F. Lorquet, M. Kleerebezem, |
| | containing the P32-cat cassette | and P. Hols. 2004. J. Bacteriol. 186: 6661- |
| | | 6666. |
| PUC18Ery | Ap^{r} Em^{r}; pUC18 derivative | van Kranenburg, R., J. D. Marugg, I. I. |
| | containing an erythromycin | van Swam, N. J. Willem, and W. M. de |
| | resistance marker | Vos. 1997. Mol. Microbiol. 24: 387-397. |
| pNZ5319 | Em^{r} Cm^{r}; pACYC184 derivative | Lambert, J. M., R. S. Bongers, and M. |
| | containing the P32-cat cassette | Kleerebezem. 2007. Appl. Environ. |
| | surrounded by lox sites | Microbiol. 73: 1126-1135. |
| pGhPcomGAluxAB | Em^{r} Ts; pG⁺host9 derivative | This application |
| | containing the llmg_pseudo_10 | |
| | (kw2_0563)::P_{comGA[MG]}-luxAB | |
| | insertion cassette | |
| pGhP32comX_{MG} | Em^{r} Ts, pG⁺host9 derivative | This application |
| | carrying comX of strain MG1363 | |
| | under the control of the constitutive | |
| | promoter P₃₂ | |
| pGhP32comX_{IO} | Em^{r} Ts, pG⁺host9 derivative | This application |
| | carrying comX of strain IO-1 under | |
| | the control of the constitutive | |
| | promoter P₃₂ | |
| pGhP32comX_{MG}- | pGhP32comX_{MG} derivative carrying | This application |
| P_{comGA[MG]}-luc | a P_{comGA[MG]}-luc fusion | |
| pGhP32comX_{IO}- | pGhP32comX_{IO} derivative carrying | This application |
| P_{comGA[IO]}-luc | a P_{comGA[IO]}-luc fusion | |
| pGEMrpsL* | Ap^{r}, pGEM ®-T easy derivative | This application |
| | carrying the rpsL* gene (strA1 | |
| | allele) | |
| pUCcomECcat | Ap^{r} Em^{r} Cm^{r}, pUC18Ery derivative | This application |
| | allowing the insertion of P₃₂-cat at | |
| | the comEC locus | |
| pGhPxylTcomX_{IO} | Em^{r} Ts, pG⁺host9 derivative | This application |
| | carrying comX of strain IO-1 | |
| | under the control of the inducible | |
| | promoter P_{xylT} from IO-1 | |

^{a} Em^{r}, Ap^{r}, Cm^{r} and Ts: erythromycin, ampicillin, chloramphenicol resistance and thermo-sensitive RepA protein, respectively.

Escherichia coli was grown with shaking at 37° C. in Lysogeny-Broth (LB) broth. Plasmids derived from pMG36e and pG⁺host9 were constructed in E. coli strains TG1 and EC1000, respectively. L. lactis and L. raffinolactis were cultivated in M17 (Becton, Dickinson, and Company), Todd Hewitt broth (THB) (Becton, Dickinson, and Company) or CDM at 30° C. without agitation. M17 and THB were supplemented with 0.5% (w/v) of glucose (M17G and THBG, respectively). Solid agar plates were prepared by adding 2% (w/v) agar to the medium. When required, 5 μg ml⁻¹ of erythromycin, 1 mg ml⁻¹ of streptomycin, and/or 10 μg ml⁻¹ of chloramphenicol were added to the medium for L. lactis and L. raffinolactis; and 250 μg ml⁻¹ of erythromycin, 250 μg ml⁻¹ of ampicillin, 10 μg ml⁻¹ of chloramphenicol for E. coli.

### Detection of Absorbance and Luminescence.

Growth (OD₆₀₀) and luciferase (Lux) activity were monitored at 10-minutes intervals in a Varioskan Flash multi-mode reader (ThermoFisher). The luciferase activity is expressed in relative light units (RLU) and the specific luciferase activity in RLU OD₆₀₀⁻¹.

### DNA Techniques and Electrotransformation

General molecular biology techniques were performed according to the instructions given by Sambrook et al. (1989). Electrotransformation of E. coli and L. lactis was performed as previously described. The electrotransformed cells of L. lactis were immediately resuspended in 1 ml of M17G and incubated for 6 hours at 30° C. Chromosomal DNAs of L. lactis were prepared as previously described. PCRs were performed with Phusion DNA polymerase (NEB) in a GeneAmp PCR system 2400 (Applied Biosystems). The primers used in this application are listed in Table 3.

TABLE 3
list of primers

| | |
|---|---|
| Primer name | Sequence (5′-3′) |

Primers used for the construction of the constitutive comX expression plasmid
pGhP32comX_{MG/IO}:

| | |
|---|---|
| BID_ComXSDLLCup | AAAAGAGCTCAATTATGAAAAAGAGG |
| BID_ComXSDLLCdown | AAAACTGC AGTTAATCATCATCTCG |
| BID_ComXSDLLLup | AAAAGAGCTCATAAAAGGAGAACTTTCC |
| BID_ComXSDLLLdown | AAAACTGCAGTCACTCTTCGTCTTC |
| BID_pMGP32UpMfeI | ATATCAATTGGTCCTCGGGATATGATAAG |
| BID_pMGTerDown | GACTTTGAACCTCAACTCC |

Primers used for the construction of the P_{comGA[MG]}-luxAB reporter strain
BLD101:

| | |
|---|---|
| BID_LuxLLCf1 | ATAGTCTCGAGTTTAAGCAATTGAATCGCTAG |
| BID_LuxLLCr1 | GCAAAAAGTTTCCAAATTTCATACTAGAATATACGCAATTTG |
| BID_LuxLLCf2 | CAAATTGCGTATATTCTAGTATGAAATTTGGAAACTTTTTGC |
| BID_LuxLLCr2 | GCGAAAGGATCCCTATTAGGTATATTCCATGTGG |
| BID_P3pseudoLLC | GCTCCCTCGAGGGCGGCTCTGTTGGATTAATATATGG |

Primers used for the construction of portable luc reporter vectors:

| | |
|---|---|
| BID_LucLLCr1 | CTTTATGTTTTTGGCGGATCTCATACTAGAATATACGCAATTTG |
| BID_LucLLCf2 | CAAATTGCGTATATTCTAGTATGAGATCCGCCAAAAACATAAAG |
| BID_LucLLCr2 | GCGAAAGGATCCTTACAATTTGGGCTTTCCG |
| BID_PcomGALLCF1* | AAAACCCGGGTTTAAGCAATTGAATCGCTAG |
| BID_PcomGALLLF1* | 5′ AAAACCCGGGAAATAAATGGCTACAAAATT |
| BID_lucR1* | AAAACGGCCGTTACAATTTGGGCTTTCCG |
| BID_luxLLLf1 | ATAGTCTCGAGAAATAAATGGCTACAAAATT |
| BID_lucLLLr1 | CTTTATGTTTTTGGCGGATCTCATACTAGACTATACGCAAATAATC |
| BID_lucLLLf2 | GATTATTTGCGTATAGTCTAGTATGAGATCCGCCAAAAACATAAAG |
| BID_lucLLLr2 | GCGAAAGGATCCTTACAATTTGGGCTTTCCG |

Primers used for the construction of pGhost-Core

| | |
|---|---|
| DD-pGhost-CoreUp | AGCTTCCTAATACAACACAATTAATATTGTGTTGTATTATTG |
| DD-pGhost-CoreDW | AATTCAATAATACAACACAATATTAATTGTGTTGTATTAGGA |

Primers used for rpsL sequencing:

| | |
|---|---|
| RpsL Univ UP | ATGCCTACAATTAACCAAT |
| RpsL Univ DN | CACCGTATTTAGAACGG |
| LR_RpsL Univ UP | ATGCCTACTATTAACCAAT |
| LR_RpsL Univ DN | TACCGTATTTAGAACGG |

Primers used for rpsL amplification:

| | |
|---|---|
| BID_LLcdacARpsL | AGTAGTATCAGCACTGACAGC |
| BID_LLlcfusARpsL | ACACCTTTGTTCTTGAAGG |

primers used for the construction of the comEC disruption mutant:

| | |
|---|---|
| BID_ComECLLCUp | AAAGAGCTCAAAATAAAAATGAAATTATGG |
| BID_ComECLLCDown | AAAGCTAGCGGGAAAAAATTGTGAATTAC |
| BID_CatUpSpeI | AAAAACTAGTGCAGTTTAAATTCGGTCCTCGG |
| BID_CatDownSpeI | AAAAACTAGTGTACAGTCGGCATTATCTCAT |

Primers used for the construction and validation of the mecA deletion mutant:

| | |
|---|---|
| BID_fgt01FmecArec | CTTTAATGATGGAATGATTG |
| BID_fgt01RVmecArec | CTATTAATCTTATCATATCCCGAGGATCCATATAACTATATGAAACC |
| BID_fgt02Fcat | TCCTCGGGATATGATAAGATTAATAG |
| BID_fgt02RVcat | TCTCATATTATAAAAGCCAGTCATTAG |
| BID_fgt03FmecArec | CTAATGACTGGCTTTTATAATATGAGACTTAGAAAAATCTAAATATGGTTG |
| BID_fgt03RVmecArec | GAAGATTTTTAATTTCAAGTGTAG |
| BID_mecAKOF | TCAGTACCGAAAAACGAATG |
| BID_mecAKORV | ATTTACCAGTTCCGTTAGG |

Primers used for the construction and validation of the ciaRH deletion mutant:

| | |
|---|---|
| BID_ciaRHUPF | TAACAATGATACAGAAGATG |
| BID_ciaRHUPRVRec | CTATTAATCTTATCATATCCCGAGGATATTTTTGTCTTGTACTAGG |
| BID_fgt02Fcat | TCCTCGGGATATGATAAGATTAATAG |
| BID_fgt02RVcat | TCTCATATTATAAAAGCCAGTCATTAG |
| BID_ciaRHDownFRec | CTAATGACTGGCTTTTATAATATGAGAGAGAGAAAAAAATTACTGAC |
| BID_ciaRHDownRV | AAAATCTGTTAGAACTGTTG |
| BID_ciaRHKODiagF | AAGATAAGGCAGTTGAAATG |
| BID_ciaRHKODiagRv | TCACCATGTGAATAAAGTCC |

Primers used for the construction and validation of the covRSdeletion mutant:

| | |
|---|---|
| BID_covRSfgt01F | CAAAAATGTGAAGCTTATC |
| BID_covRSfgt01RVRec | CTATTAATCTTATCATATCCCGAGGATGCATAATTCGATTTC |
| BID_fgt02Fcat | TCCTCGGGATATGATAAGATTAATAG |
| BID_fgt02RVcat | TCTCATATTATAAAAGCCAGTCATTAG |
| BID_covRSfgt03FRec | TAATGACTGGCTTTTATAATATGAGACTATTTATCTGCTCATTTC |
| BID_covRSfgt03RV | GAGCTTTTTTCAAATCTTC |
| BID_covRSKOFdiag | GAAGTGATGAATGAGATG |
| BID_covRSKORVdiag | CTTTCTCATCAATTGAGAC |

Primers used for the construction and validation of the clpC deletion mutant:

| | |
|---|---|
| BID_clpCUPF | CTTTGGGTTCTAATTTATC |
| BID_clpCUPRVRec | CTATTAATCTTATCATATCCCGAGGACGTTGGTGTATATTTTAC |
| BID_fgt02Fcat | TCCTCGGGATATGATAAGATTAATAG |
| BID_fgt02RVcat | TCTCATATTATAAAAGCCAGTCATTAG |
| BID_clpCDownFRec | CTAATGACTGGCTTTTATAATATGAGATAGAAATAAAGGAAAGGAC |
| BID_clpCDownRV | TTGCTTTAAGGATAGTTTC |
| BID_clpCFdiag | AGAAGCCAATAATGACGATG |
| BID_clpCRVdiag | AGAATTCTGATGATGCACAGTC |

Primers used for the construction of the inducible comX expression plasmid
pGhPxylTcomX_{IO}:

| | |
|---|---|
| FT_ | AGCGCCGCGGTGGGATCCTCTAGAGTC |
| pGhPxylcomXIOsacllrv | |
| FT_pGhPxylcomX | CTGCAGGCATGCACATCATCAACTTGAAGGG |
| FT_PxylTIOsacIIfw | CCCACCGCGGTGGAGATACGAACAAATTAG |
| FT_PxylTIOrv | GATAGTAACTCCTTAATTTTTATTTGC |
| FT_comXIOrecfw | GCAAATAAAAATTAAGGAGTTACTATCATGACATATTACTTGGAAGAAGAGGAT |
| | TTTG |
| FT_comXIOrecrv | CCTTCAAGTTGATGATGTGCATGCCTGCAGTCACTCTTCGTCTTC |

Primers used for the construction and validation of the SL12653-comX deletion
mutant

| | |
|---|---|
| FT_comXlocusfw | TGACCATGTTACACAAGCCTATATCCT |
| FT_comXrecrv | CGCCCTTATGGGATTTATCTTCCTTACTTCGTTTCTTTGCATAACTTCGTCTTA |
| | AT |
| Uplox66 | TAAGGAAGATAAATCCCATAAGG |
| Dnlox71 | TTCACGTTACTAAAGGGAATGTA |
| FT_comXrecfw | TCTACATTCCCTTTAGTAACGTGAACCATGACCATTTTATAGGTTTAGATGTTT |
| | ATG |
| AR_comxDNspecR | CGGTGTTCCTCCATATATCTACGC |
| FT_PxylcomXfw | CGCTAAACTCAACAGGTGATCCGATTG |

Construction of Plasmid pGhP32comX_{MG}

As a representative of the cremoris subspecies, the comX gene from the laboratory strain MG1363 was initially chosen. ComX proteins of this subspecies are highly conserved with at least 98% of identity. The comX gene was amplified by PCR using primers BID_ComXSDLLCup/BID_ComXSDLLCdown and inserted into plasmid pMG36eT under the control of the constitutive P₃₂ promoter by Sacl/Pstl cloning, yielding plasmid pMGP32comX_{MG}. The P₃₂-comX_{MG} fusion from pMGP32comX_{MG} was amplified by PCR with primers BID_pMGP32UpMfel/BID_pMGTerDown, digested by Mfel/Kpnl, and cloned in the EcoRI/Kpnl-digested thermosensitive pG⁺host9 vector. The resulting plasmid was named pGhP32comX_{MG}.

Construction of plasmid pGhP32comX_{IO}

As a representative of the lactis subspecies, the comX gene from the IO-1 strain was chosen. The comX gene was amplified by PCR using primers BID_ComXSDLLLup/BID_ComXSDLLLdown and inserted into plasmid pMG36eT under the control of the constitutive P₃₂ promoter by Sacl/Pstl cloning, yielding plasmid pMGP32comX_{IO}. The P₃₂-comX_{IO} fusion from pMGP32comX_{IO} was amplified by PCR with primers BID_pMGP32UpMfel/BID_pMGTerDown, digested by Mfel/Kpnl, and cloned in the EcoRI/Kpnl-digested thermosensitive pG⁺host9 vector. The resulting plasmid was named pGhP32comX_{IO}.

Construction of plasmid pGhost-Core

The Core part of the resolution site (IRS) recognized by the Tnpl recombinase from Tn4430 was assembled by using the complementary primers DD-pGhost-CoreUp/DD-pGhost-CoreDW. The resulting DNA fragment was cloned between HindIII and EcoRI sites in plasmid pG⁺host9. The resulting plasmid, named pGhost-Core, was transformed in E. coli harbouring plasmid pGIV004 (Tnpl⁺) for obtaining multimeric forms (Vanhooff V, Galloy C, Agaisse H, Lereclus D, Révet B, Hallet B. Mol Microbiol. 2006 May; 60 (3): 617-29).

### Construction of P_{comGA[MG]-Lux}AB Reporter Strain BLD101

The P_{comGA[MG]} promoter was amplified by PCR from chromosomal DNA of L. lactis MG1363 (identical nucleotide sequence between MG1363 and KW2) with primers BID_LuxLLCf1/BID_LuxLLCr1 (PCR1 product). The luxAB genes were amplified by PCR from plasmid pJIM4900 with primers BID_LuxLLCf2/BID_LuxLLCr2 (PCR2 product). The P_{comGA[MG]}-luxAB fusion was created by overlapping PCR using PCR1 and PCR2 products and primers BID_LuxLLCf1/BID_LuxLLCr2. The resulting fusion was cloned in plasmid PSEUDOPusp45GFP using restriction enzymes Xhol and BamHI, yielding plasmid PSEUDOPusp45PcomGAluxAB. In order to remove the Pusp45 promoter, the entire vector except the Pᵤₛₚ₄₅ promoter was amplified by inverse PCR with primers BID_P3pseudoLLC/BID_LuxLLCf1 and self-ligated after Xhol digestion, leading to plasmid PSEUDOPcomGAluxAB. The insertion cassette IImg_pseudo_10::P_{comGA[MG]} luxAB was excised from plasmid pSEUDOPcomGAluxAB and cloned into the pG⁺host9 thermosensitive vector using restriction enzymes Kpnl/EagI. The resulting plasmid pGhPcomGAluxAB was then electro-transformed in strain KW2 and used to integrate the P_{comGA[MG]}-luxAB cassette at locus kw2_0563 (llmg_pseudo_10 in MG1363) by double homologous recombination, resulting in the reporter strain KW2 kw2_0563::P_{comGA[MG]}-luxAB (strain BLD101).

### Construction of Portable Luc Reporter Systems

The P_{comGA[MG]} promoter was amplified by PCR from chromosomal DNA of L. lactis MG1363 with primers BID_LuxLLCf1/BID_LucLLCr1 (PCR1 product). The luc gene was amplified by PCR from plasmid pXL with primers BID_LucLLCf2/BID_LucLLCr2 (PCR2 product). The P_{comGA[MG]} luc fusion was created by overlapping PCR using PCR1 and PCR2 products and primers BID_LuxLLCf1/BID_LucLLCr2. The resulting fusion was cloned in plasmid PSEUDOPusp45GFP using restriction enzymes Xhol and BamHI, yielding plasmid pSEUDOPusp45PcomGAluc. In order to remove the Pusp45 promoter, the entire vector except the Pusp45 promoter was amplified by inverse PCR with primers BID_P₃pseudoLLC/BID_LuxLLCf1 and self-ligated after Xhol digestion, leading to plasmid PSEUDOPcomGAluc. The reporter cassette P_{comGA[MG]}-luc was amplified by PCR from PSEUDOPcomGAluc (primers BID_PcomGALLCF1*/BID_lucR1*) and cloned between XmaI and EagI into the pGhP32comX_{MG} plasmid. The resulting reporter plasmid was named pGhP32comX_{MG}-P_{comGA[MG]}-luc.

The P_{comGA[IO]} promoter was amplified from the IO-1 chromosome (primers BID_luxLLLf1/BID_lucLLLr1) and the luciferase gene (luc) was amplified from plasmid pXL (primers BID_lucLLLf2/BID_lucLLLr2). The cassette P_{comGA[IO]}-luc was created by overlapping PCR with primers BID_luxLLLf1/BID_lucLLLr2. The cassette P_{comGA} [IO]-luc was then amplified from the overlapping PCR product with primers BID_PcomGALLLF1*/BID_lucR1* for XmaI/EagI cloning into pGhP32comX_{IO}. The resulting reporter plasmid was named pGhP32comX_{IO}-P_{comGA[IO]}-luc.

Isolation of a rpsL Mutant Conferring Resistance to Streptomycin

Spontaneous streptomycin-resistant MG1363 clones were isolated on 1 mg ml⁻¹ streptomycin-containing plates. After the sequencing of the rpsL gene with primers RpsL Univ UP/RpsL Univ DN, one spontaneous mutant resulting in a mutation (K561) into the ribosomal protein S12 that was previously shown to confer resistance to streptomycin was selected (FIG. 6). A 3.7-kb fragment containing the rpsL mutated gene (strA1 allele) was amplified by PCR with primers BID_LLcdacARpsL/BID_LLIcfusARpsL and cloned into the pGEM®-T easy vector (Promega), yielding plasmid pGEMrpsL*. This plasmid was used as template to generate the 3.7-kb PCR product with primers BID_LLcdacARpsL/BID_LLIcfusARpsL that was used as donor DNA in natural transformation assays of strain KW2.

### Standard Natural Transformation Assay

The BLD101 reporter strain carrying the pGhP32comX_{MG} plasmid (BLD101 [pGhP32comX_{MG}]) was grown overnight in M17G at 30° C. Then, 1.5 ml of the pre-culture was diluted in 8.5 ml of fresh M17G medium to restart the culture. After 2 hours of growth, cells were washed twice in distilled water and OD₆₀₀ was adjusted to 0.05 in CDM containing erythromycin (5 μg ml⁻¹) and supplemented with either 5% (v/v) glycerol or 5% (w/v) mannitol used as potential osmo-stabilizers. Typically, 5 μg of DNA was added in 300 μl of inoculated medium and the culture was further incubated during 6 hours at 30° C. Cells were then spread on M17G agar plates supplemented with appropriate antibiotics and CFUs were counted after 48 hours of incubation. The transformation frequency was calculated as the number of antibiotic-resistant CFU ml⁻¹ divided by the total number of viable CFU ml⁻¹. In the case of streptomycin-resistant transformants, antibiotic-resistant CFU ml⁻¹ corresponds to the number of transformants obtained in presence of DNA less the number of spontaneous transformants obtained in conditions where no DNA is added in the culture. The transfer of the mutation conferring streptomycin resistance was confirmed by DNA sequencing of the rpsL gene after its amplification by PCR using primers RpsL Univ UP/RpsL Univ DN.

Disruption of comEC by Natural Transformation

A comEC-containing DNA fragment of ˜3.2 kb was amplified by PCR with primers BID_ComECLLCUp/BID_ComECLLCDown. Then, the PCR product was digested by Sacl/NheI and cloned into the Sacl/Xbal-digested suicide plasmid pUC18Ery (van Kranenburg et al., 1997), yielding plasmid pUCcomEC. To generate a comEC disruption cassette that allows the selection of double crossing-over recombinants, the P₃₂-cat fusion conferring resistance to chloramphenicol was cloned in the middle of the comEC gene. For this purpose, the P₃₂-cat cassette was amplified by PCR from plasmid pNZ5319 (Lambert et al., 2007, Appl. Environ. Microbiol. 73:1126-1135) with primers BID_CatUpSpel/BID_CatDownSpel. The amplification product was digested by Spel and cloned into the Xbal-digested pUCcomEC, yielding plasmid pUCcomECcat. This suicide plasmid was used to generate high quantity of donor DNA by PCR amplification for comEC disruption by natural transformation. The insertion of the P₃₂-cat cassette in the comEC gene of KW2 transformants was validated by PCR (primers in Table 3).

Deletion of mecA, ciaRH, covRS, and clpC Genes by Natural Transformation

The mecA, ciaRH, covRS, and clpC genes were similarly inactivated by the exchange of their ORFs by the P₃₂-cat cassette using double crossing-over events. For this purpose, overlapping PCR products containing the P₃₂-cat cassette flanked by two recombination arms of ˜1.5 kb (upstream and downstream homologous regions) were generated as previously reported. Briefly, upstream, downstream, and P₃₂-cat fragments were separately amplified by PCR, purified, mixed in equimolar concentration, and assembled by overlapping PCR by using the most external primers (see list of primers in Table 3). 5 μg of the obtained overlapping PCR product was used as donor DNA for natural transformation of strain BLD101 [pGhP32comX_{MG}]. The correct insertion of the P₃₂-cat cassette in each targeted locus of the KW2 transformants was validated by PCR (see list of primers in Table 3). To obtain the final mutant strains, the thermosensitive vector pGhP32comX_{MG} was cured by growing the strains overnight at 37° C. without erythromycin. The cultures were subsequently diluted and plated on M17G agar without erythromycin at 30° C. The resulting colonies were streaked in parallel on M17G plates with and without erythromycin. Absence of plasmid pGhP32comX_{MG} in Ery^{S} clones was validated by PCR.

### Induction of Natural Competence in Lactococcus Raffinolactis

Wild-type Lactococcus raffinolactis (i.e., L. raffinolactis strains which have not been previously engineered for the overproduction of the comX gene) were grown overnight in M17G at 30° C. 1.5 ml of the pre-culture was diluted in 8.5 ml of fresh M17G medium to restart the culture. After 2 hours of growth, cells were washed twice in distilled water and OD₆₀₀ was adjusted to 0.05 in CDM supplemented with either 5% (v/v) glycerol or 5% (w/v) mannitol used as potential osmo-stabilizers. 15 μg of plasmid pGhost-Core was added in 300 μl of inoculated medium and the culture was further incubated during 6 hours at 30° C. Cells were then spread on M17G agar plates supplemented with appropriate antibiotics and CFUs were counted after 48 hours of incubation. The transformation frequency was calculated as the number of antibiotic-resistant CFU ml⁻¹ divided by the total number of viable CFU ml⁻¹.

Natural Competence in Lactococcus lactis Subsp Lactis SL12651 and SL12653 Strains

The L. lactis subsp. lactis SL12653 and 12651 strains were grown overnight at 30° C. Cells were washed twice in distilled water and OD600 was adjusted to 0.05 in M17G. Typically, 5 μg of donor DNA was added in 200 μl of inoculated medium (25 μg/ml) and the culture was further incubated during 24 hours at 30° C. Cells were then spread on M17G agar plates supplemented with appropriate antibiotics and CFUs were counted after 48 hours of incubation at 30° C. The transformation frequency calculated exactly as described above (see Standard natural transformation assay).

The same experiments were done in SL12653 with various concentrations of donor DNA (0.5, 2.5, 5 and 25μ g/ml)

Construction of plasmid pGhPxyITcomXIO

As a representative of the lactis subspecies, the comX gene and the promoter of the xyIT gene from the IO-1 strain were chosen. The comX gene was amplified by PCR using primers FT_comXIOrecfw and FT_comXIOrecrv (PCR1), both containing overlapping sequences. The xyIT promoter region was amplified by PCR using primers FT_PxylTIOsacllfw and FT_PxylTIOrv (PCR2). The carrying vector was amplified from plasmid pGhP32comX_{MG} and amplified by PCR using primers FT_pGhPxylcomXIOsaclIrv and FT_pGhPxylcomX (PCR3). The three PCR products were purified, mixed in an equimolar concentration and assembled by overlapping PCR using the most external primers, containing a Sacll restriction site. The amplification product was digested by Sacll and self-ligated. The resulting plasmid was named pGhPxyITcomX_{IO}.

Transformation Assay in SL12653 Mutants Deleted for the comX Gene

The comX gene of SL12653 was inactivated by exchange of their ORF by the P₃₂-cat cassette using double crossing-over events. For this purpose, overlapping PCR products containing the P₃₂-cat cassette flanked by two recombination arms of ˜1.5 kb (upstream and downstream homologous regions) were generated as previously reported. Briefly, upstream, downstream, and the P₃₂-cat fragments were separately amplified by PCR, purified and mixed in equimolar concentration, and assembled by overlapping PCR by using the most external primers (see primers in Table 3). 5 μg of the obtained PCR product was used as donor DNA for natural transformation of strain SL12653 [pGhPxyITcomX_{IO}] (ComX+). The correct insertion of the P₃₂-cat cassette in the targeted locus of SL12653 transformants was validated by PCR (see primers in Table 3). To obtain the final mutant strains, the thermosensitive vector pGhPxylTcomX_{IO} was cured by growing the strains overnight at 37° C. without erythromycin. The cultures were subsequently diluted and plated on M17G agar without erythromycin at 30° C. The resulting colonies were streaked in parallel on M17G plates with and without erythromycin. Absence of plasmid pGhPxyITcomX_{IO} in Ery^{S} clones was validated by PCR. Thus, 3 AcomX clones of SL12653 were obtained.

### Xylose-Induced Natural Transformation in SL12653.

The L. lactis subsp. lactis SL12653 [pGhPxylTcomX_{IO}] was grown overnight at 30° C. Cells were washed twice in distilled water and OD600 was adjusted to 0.05 in M17 supplemented with 1% (w/v) xylose. Typically, 5 μg of DNA was added in 200 μl of inoculated medium and the culture was further incubated during 24 hours at 30° C. Cells were then spread on M17G agar plates supplemented with appropriate antibiotics and CFUs were counted after 48 hours of incubation at 30° C. The transformation frequency was calculated exactly as described above (see Standard natural transformation assay).

All publications mentioned in the above specification are herein incorporated by reference. Various modifications and variations of the described present invention will be apparent to those skilled in the art without departing from the scope and spirit of the present invention. Although the present invention has been described in connection with specific preferred embodiments, it should be understood that the invention as claimed should not be unduly limited to such specific embodiments. Indeed, various modifications of the described modes for carrying out the invention which are obvious to those skilled in molecular biology, biochemistry, microbiology, bacteriology, or related fields are intended to be within the scope of the following claims.

### REFERENCES

Bachmann, H., W. L. de, M. Kleerebezem, and J. E. van Hylckama Vlieg. 2010. Time-resolved genetic responses of Lactococcus lactis to a dairy environment. Environ. Microbiol. 12:1260-1270.
Campbell, E. A., S. Y. Choi, and H. R. Masure. 1998. A competence regulon in Streptococcus pneumoniae revealed by genomic analysis. Mol. Microbiol. 27:929-939
David, B., Radziejwoski, A., Toussaint, F., Fontaine, L., Henry de Frahan, M., Patout,
C.,van Dillen, S., Boyaval, P., Horvath, P., Fremaux, C. and P. Hols. 2017. Natural DNA transformation is functional in Lactococcus lactis subsp. cremoris KW2. Appl. Environ. Microbiol. 83 (16): 1-17
Ercan, O., M. Wels, E. J. Smid, and M. Kleerebezem. 2015. Genome-wide transcriptional responses to carbon starvation in nongrowing Lactococcus lactis. Appl. Environ. Microbiol. 81:2554-2561.
Fontaine, L., C. Boutry, M. H. de Frahan, B. Delplace, C. Fremaux, P. Horvath, P. Boyaval, and P. Hols. 2010. A novel pheromone quorum-sensing system controls the development of natural competence in Streptococcus thermophilus and Streptococcus salivarius. J. Bacteriol. 192:1444-1454.
Lee, M. S. and D. A. Morrison. 1999. Identification of a new regulator in Streptococcus pneumoniae linking quorum sensing to competence for genetic transformation. J. Bacteriol. 181:5004-5016.
Luo, P. and D. A. Morrison. 2003. Transient association of an alternative sigma factor, ComX, with RNA polymerase during the period of competence for genetic transformation in Streptococcus pneumoniae. J. Bacteriol. 185:349-358
Martin-Galiano and de la Campa 2003. High-Efficiency Generation of Antibiotic-Resistant Strains of Streptococcus pneumoniae by PCR and Transformation. Antimicrob Agents Chemother. 47 (4): 1257-1261
Peterson, S. N., C. K. Sung, R. Cline, B. V. Desai, E. C. Snesrud, P. Luo, J. Walling, H. Li, M. Mintz, G. Tsegaye, P. C. Burr, Y. Do, S. Ahn, J. Gilbert, R. D. Fleischmann, and D. A. Morrison. 2004. Identification of competence pheromone responsive genes in Streptococcus pneumoniae by use of DNA microarrays. Mol. Microbiol. 51:1051-1070
Terzaghi and Sandine 1975. Improved Medium for Lactic Streptococci and Their Bacteriophages. Applied Microbiology 29 (6): 807-813
Todd, E. W., and L. F. Hewitt. 1932. A new culture medium for the production of antigenic streptococcal haemolysin. J. Pathol. Bacteriol. 35:973-975
Updyke, E. L., and M. I. Nickle. 1954. A dehydrated medium for the preparation of type specific extracts of group A streptococci. Appl. Microbiol. 2:117-118
Sambrook, J., E. F. Fritsch, and T. Maniatis. 1989. Molecular cloning: a laboratory manual.
Sissler, M., Delorme, C., Bond, J., Dusko Ehrlich, S., Renault, P. and C. Francklyn. 1999. An aminoacyl-tRNA synthetase paralog with a catalytic role in histidine biosynthesis. Proc. Natl. Acad. Sci. 96:8985-8990
van Kranenburg, R., J. D. Marugg, I. I. van Swam, N. J. Willem, and W. M. de Vos. 1997.Molecular characterization of the plasmid-encoded eps gene cluster essential for exopolysaccharide biosynthesis in Lactococcus lactis. Mol. Microbiol. 24:387-397.
Ward, L. J., J. C. Brown, and G. P. Davey. 1998. Two methods for the genetic 5 differentiation of Lactococcus lactis ssp. lactis and cremoris based on differences in the 16S rRNA gene sequence. FEMS Microbiol Lett. 166:15-20
Wegmann, U., M. O'Connell-Motherway, A. Zomer, G. Buist, C. Shearman, C. Canchaya, M. Ventura, A. Goesmann, M. J. Gasson, O. P. Kuipers, S. D. van, and J. Kok. 2007. Complete genome sequence of the prototype lactic acid bacterium Lactococcus lactis subsp. cremoris MG1363. J. Bacteriol. 189:3256-3270.
Wydau, S., R. Dervyn, J. Anba, E. S. Dusko, and E. Maguin. 2006. Conservation of key elements of natural competence in Lactococcus lactis ssp. FEMS Microbiol. Lett. 257:32-42.

1-17. (canceled)

18. A strain of the Lactococcus genus obtained or obtainable by a method for transforming a strain of the Lactococcus genus with an exogenous DNA polynucleotide comprising the steps of:
(a) providing a strain of the Lactococcus genus, wherein said strain is transformable through natural competence;
(b) modulating the production of a ComX protein in said strain;
(c) contacting said strain of step (b) with an exogenous DNA polynucleotide in a medium and incubating the resulting mixture for integration of the exogenous DNA polynucleotide into the genome of said strain; and
(d) selecting a strain which has integrated the exogenous DNA polynucleotide into its genome.

19. The strain according to claim 18, wherein the step of modulating the production of a ComX protein is performed by expressing a comX gene in said strain or increasing the expression of a comX gene in said strain.

20. The strain according to claim 19, wherein said comX gene is an exogenous comX gene.

21. The strain according to claim 20, wherein said exogenous comX gene is transferred into said strain by conjugation, transduction, or transformation.

22. The strain according to claim 19, wherein said comX gene is the endogenous comX gene of said strain.

23. The strain according to claim 22, wherein the method comprises carrying out step (b) and then carrying out step (c) or comprises carrying out step (b) and step (c) simultaneously.

24. The strain according to claim 18, wherein said ComX protein has the amino acid sequence of SEQ ID NO: 2, SEQ ID NO:4, SEQ ID NO:6, SEQ ID NO:8, SEQ ID NO: 10, SEQ ID NO:12, SEQ ID NO: 14, SEQ ID NO:16, SEQ ID NO: 18, SEQ ID NO: 20 or SEQ ID NO:22 or an amino acid sequence having at least 90%, at least 95%, at least 97%, or at least 99% identity to the amino acid sequence of SEQ ID NO: 2, SEQ ID NO: 4, SEQ ID NO:6, SEQ ID NO:8, SEQ ID NO: 10, SEQ ID NO:12, SEQ ID NO: 14, SEQ ID NO:16, SEQ ID NO:18, SEQ ID NO:20 or SEQ ID NO:22 or an amino acid sequence having at least 90%, at least 95%, at least 97%, or at least 99% similarity to the amino acid sequence of SEQ ID NO: 2, SEQ ID NO:4, SEQ ID NO:6, SEQ ID NO: 8, SEQ ID NO: 10, SEQ ID NO:12, SEQ ID NO: 14, SEQ ID NO:16, SEQ ID NO: 18, SEQ ID NO:20 or SEQ ID NO:22.

25. The strain according to claim 19, wherein said comX gene has the nucleotide sequence of SEQ ID NO:1, SEQ ID NO:3, SEQ ID NO:5, SEQ ID NO:7, SEQ ID NO:9, SEQ ID NO:11, SEQ ID NO:13, SEQ ID NO:15, SEQ ID NO:17, SEQ ID NO: 19, SEQ ID NO: 21 or a nucleotide sequence having at least 90%, at least 95%, at least 97% or at least 99% identity to the nucleotide sequence of SEQ ID NO:1, SEQ ID NO:3, SEQ ID NO: 5, SEQ ID NO:7, SEQ ID NO:9, SEQ ID NO: 11, SEQ ID NO: 13, SEQ ID NO:15, SEQ ID NO:17, SEQ ID NO:19, SEQ ID NO:21.

26. The strain according to claim 18, wherein said medium of step (c) is a chemically defined medium.

27. The strain according to claim 18, wherein prior to step (c) said strain is incubated in a pre-culture medium.

28. The strain according to claim 18, wherein said strain is incubated with the exogenous DNA polynucleotide for around 4-8 hours at around 30° C. and said medium of step (c) is supplemented with an osmo-stabilizer.

29. The strain according to claim 18, wherein said strain of the Lactococcus genus of step (a) is a strain of the Lactococcus raffinolactis species or a strain of the Lactococcus lactis species.

30. The strain according to claim 18, wherein said exogenous DNA polynucleotide used in step (c) is from a strain of the same species.
